(19) United States
(12) Patent Application Publication
(10) Pub. No.: US 2019/0314312 A1
(43) Pub. Date: Oct. 17, 2019

(54) COMBINATION THERAPY TO INCREASE ENDOGENOUS NITRIC OXIDE (NO) SYNTHESIS

(71) Applicant: Yeda Research and Development Co. Ltd., Rehovot (IL)

(72) Inventors: Ayelet EREZ, Moshav Bnei Zion (IL); Noa STETTNER, Rehovot (IL)

(73) Assignee: Yeda Research and Development Co. Ltd., Rehovot (IL)

(21) Appl. No.: 16/316,639

(22) PCT Filed: Jul. 11, 2017
(86) PCT No.: PCT/IL2017/050791

§ 371 Date: Jan. 10, 2019

(30) Foreign Application Priority Data
Jul. 11, 2016 (IL) 246722

(51) Int. Cl. A61K 31/198; A61K 31/366; A61P 1/00; A61P 35/00

(52) U.S. Cl. CPC A61K 31/198; A61P 35/00; A61P 1/00; A61K 31/366

(57) ABSTRACT

Provided are uses of a therapeutically effective amount of citrulline for the manufacture of a medicament for treatment of a subject having an inflammatory bowel disease (IBD) and/or colon cancer. Also provided are pharmaceutical compositions comprising a therapeutic effective amount of citrulline and a therapeutically effective amount of a flavonoid for the treatment of an inflammatory bowel disease (IBD) and/or colon cancer and kits comprising same.

### FIELD AND BACKGROUND OF THE INVENTION

The present invention, in some embodiments thereof, relates to using citrulline and/or with combination of a flavonoid for the manufacture of a medicament for treatment of a subject having an inflammatory bowel disease (IBD) and/or colon cancer.

Inflammatory bowel diseases (IBDs) such as Crohn's disease and ulcerative colitis are chronic inflammatory disorders of the intestinal tract. Multiple factors including genetic predisposition, environment, gut microbiota, altered barrier function of the intestinal lining and dysregulated immune response are key elements involved in IBD pathogenesis. While the precise causes of IBD are still unknown, prior research postulates that IBD arises mainly from the interplay between appropriate and excessive immune responses, generated in a cell specific manner by the tissue and the systemic immune system, with Nitric oxide (NO) playing an important role in both^{1,2}.

Nitric oxide (NO), an important signaling molecule, is a homeostatic regulator of gastrointestinal integrity by maintaining perfusion, epithelial and vascular permeability and gut motility. In addition, NO signaling is important for the host immune response and tissue repair. Although substantial increase in NO signaling has been implicated in the pathogenesis of human IBD, the specific cellular origin of NO and the exact roles of epithelial and immune cell-derived NO in intestinal inflammation remain unclear (Boughton-Smith, N. K. et al. 1993. The Lancet 342, 338-e2; Kolios, G. et al., 2004. Immunology 113, 427-437; Middleton, S. J. et al., 1993. The Lancet 341, 465-466; Suschek, C. V. et al., 2004. Curr. Mol. Med. 4, 763-775). A primary reason for the limited understanding of the role of cell- and context-dependent NO signaling in IBD is the redundancy of the three isoforms of nitric oxide synthase (NOS): endothelial (eNOS), neuronal (nNOS), and inducible (iNOS). The constitutively expressed isoforms eNOS and nNOS generate low levels of NO that are essential for normal physiological functions; whereas iNOS generates high amounts of NO in response to immunogenic and inflammatory stimuli. The NOS isoforms are often co-expressed in multiple cell types, which prevent dissection of the cell specific NO contributions to disease pathogenesis in both in vitro and in vivo models (Knowles, R. G. et al., 1994. Biochem. J. 298, 249-258). These obstacles have precluded implementation of NO-related therapies, necessitating a translational model system that overcomes these limitations (Boughton-Smith, N. K. et al. 1993, Supra). All three NOS isoforms use L-arginine as substrate for NO synthesis. Argininosuccinate lyase (ASL) is the only mammalian enzyme that can endogenously generate L-arginine, the substrate for NO. As L-arginine is a semi-essential amino acid, in arginine-deficient states, such as intestinal inflammation, ASL has a key role in maintaining arginine homeostasis at the tissue level. The present inventors have previously shown that ASL is not only required for the synthesis of arginine, but also for the structural stability of a protein complex containing NOS that is critical for generation of NO, and that decreasing its levels results in decreased NO production (Erez, A. et al., 2011. Nat. Med. 17, 1619-1626). Loss of ASL leads to metabolic restriction of arginine for all NOS-derived NO. In addition, by generating an As1 conditional knockout (As1 cko) mouse in enterocytes, the present inventors showed that newborn pups have increased propensity to develop necrotizing enterocolitis⁷.

Sahu B D., et al., 2016 (J. Nutr. Biochem, 28:171-182) shows that fisetin significantly reduces pro-inflammatory cytokine in vitro from lipopolysaccharide (LPS)-stimulated mouse primary peritoneal macrophages and states that fisetin may be a promising candidate in treatment of inflammatory bowl disease.

Additional background art include Lee E H et al., 2013 (J. Pediatr. Gastroenterol. Nutr. 57: 570-575); Kimura H., et al., 1998 (Gut; 42: 180-187); Rachmilewitz D., et al., 1998 (Am. J. Gastroenterol. 93: 409-412); Godkin A J., et al., 1996 (Eur. J. Clin. Invest. 26: 867-872); Rachmilewitz D., et al., 1995 (Gut; 36: 718-723); Ribbons K A et al., 1995 (Gastroenterology, 108: 705-711); US 20010056068 to CHWALISZ, KRISTOF et al.

### SUMMARY OF THE INVENTION

According to an aspect of some embodiments of the present invention there is provided a use of a therapeutically effective amount of citrulline for the manufacture of a medicament for treatment of a subject having an inflammatory bowel disease (IBD) and/or colon cancer.

According to an aspect of some embodiments of the present invention there is provided a method of treating inflammatory bowel disease (IBD) and/or colon cancer comprising administering to a subject in need thereof a therapeutically effective amount of citrulline, thereby treating the subject.

According to an aspect of some embodiments of the present invention there is provided a use of a therapeutically effective amount of citrulline and a therapeutically effective amount of a flavonoid for the manufacture of a medicament for treatment of a subject having an inflammatory bowel disease (IBD) and/or colon cancer.

According to an aspect of some embodiments of the present invention there is provided a method of treating inflammatory bowel disease (IBD) and/or colon cancer comprising administering to a subject in need thereof a therapeutically effective amount of citrulline and a therapeutically effective amount of a flavonoid, thereby treating the subject.

According to an aspect of some embodiments of the present invention there is provided a pharmaceutical composition comprising a therapeutic effective amount of citrulline and a therapeutically effective amount of a flavonoid for the treatment of an inflammatory bowel disease (IBD) and/or colon cancer.

According to an aspect of some embodiments of the present invention there is provided a kit for the treatment of an inflammatory bowel disease (IBD) and/or colon cancer comprising a first container packaging a first pharmaceutical composition comprising a therapeutic effective amount of citrulline, and a second container packaging a second pharmaceutical composition comprising therapeutically effective amount of a flavonoid.

According to some embodiments of the invention, the citrulline is co-administered with a therapeutically effective amount of a flavonoid.

According to some embodiments of the invention, the therapeutically effective amount of the citrulline and/or of the flavonoid upregulates the level of nitric oxide in enterocyte cells of the subject.

According to some embodiments of the invention, the therapeutically effective amount of the citrulline and/or of the flavonoid upregulates the level of Argininosuccinate Lyase (ASL) in the entrerocyte cells.

According to some embodiments of the invention, the flavonoid is naturally-occurring flavonoid.

According to some embodiments of the invention, the naturally-occurring flavonoid is fisetin.

According to some embodiments of the invention, the IBD comprises ulcerative colitis.

According to some embodiments of the invention, the IBD comprises Crohn's disease.

According to some embodiments of the invention, the disease is colon cancer.

According to some embodiments of the invention, the method further comprising administering to the subject an agent which downregulates Argininosuccinate Lyase (ASL) in immune cells of the subject.

According to some embodiments of the invention, the immune cells comprise macrophages.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I demonstrate that reduced NO production by the enterocytes increases colitis severity. FIG. 1A-A schematic illustration of the colitis experimental model using mice with deletion of As1 in the hematopoietic system—Vav1^{Cre}: As1^{f/f}, and CD11c^{Cre}:As1^{f/f} and in mice with deletion of As1 in the epithelial cells—Villin^{Cre}: As1^{f/f}. The magnified cells in circles represent the CKO in enterocytes by Villin^{Cre}:As1^{f/f}(upper right) and the CKO in immune cells in general by VavCre^{+/-} and specifically in macrophages by CD11cCre^{+/-} (lower left). FIGS. 1B-D—Colitis was induced by DSS according to the published protocol in As1^{f/f};Villin cre^{tg/+} and in control As1^{f/f} mice fed on arginine free diet. As1^{f/f} Villin Cre^{+/-} had increased colitis severity in comparison to control mice as demonstrated by a significant weight loss (FIG. 1B), a higher endoscopic colitis score (n>18 in each group; FIG. 1C) and a higher histological score (n<6 in each group; FIG. 1D). FIG. 1E—A T2 map shows increased relaxation time reflecting the increased colitis severity in the As1^{f/f} Villin Cre^{+/-} as compared to control mice as demonstrated by the color gradient (blue colors—mild, red—severe). The lower panel shows quantification of the images using Matlab software. (n≥11 in each group), * P<0.05, ** P<0.005. FIG. 1F—shows a higher mortality rate. The right panels in FIGS. 1C and 1D show a representative image of the colon taken endoscopically in FIG. 1C and colon cross sections stained by H&E in FIG. 1D showing severe edema and destruction of the colon crypts in the As1^{f/f} Villin Cre^{+/-} mice. Bar, 50 μm. FIG. 1G—Upregulation of As1 in the liver following colitis induction as demonstrated at the RNA level using smFISH for As1. The small white dots are single mRNA molecules of As1. It is noted that there are more transcription sites on the villin compare to the WT, bigger and brighter dots pointed by the arrows in the nucleus, indicating increased activity. Blue are DAPI-stained nuclei, red is Phalloidin membrane staining. Scale bar is 5 μm, Image is a maximal projection of 6 optical sections spaced 0.3 μm apart. * P<0.05, ** P<0.005, ***P<0.0005. FIG. 1H—NO₂ levels measured from distal colon section by dedicated HPLC system, shows significant reduction in the NO₂ levels after colitis induction in As1^{f/f} Villin Cre^{+/-} mice compare to control (n=7 in each group). * P<0.05, ** P<0.005. FIG. 1I—Human data taken from GEO data set analysis for RNA sequencing taken from 359 people, shows significant upregulation of SL expression in patients suffering from Crohn's disease and ulcerative colitis (UC), compared to normal.

FIGS. 2A-H demonstrate reduced MQ activation and colitis severity in As1^{f/f} Vav Cre^{+/-} Colitis was induced by DSS in As1^{f/f};Vav cre^{+/-} and in control As1^{f/f} mice. FIG. 2A—Endoscopic evaluation on days 12 after colitis induction shows significant improvement in the colitis score of the As1^{f/f} Vav Cre^{+/-} group compared to the control group (n≥15 in each group). The right panel shows a representative image of the colon taken by colonoscopy. FIG. 2B—A T2 map demonstrating increased relaxation time in the control group as compare to As1^{f/f};Villin cre^{tg/+}. The right panel shows quantification of the images using Matlab software. FIG. 2C—Anemia developed in the control group after colitis induction, as shown by red blood cells (RBC), hemoglobin (HgB) and hematocrit (HCT) reduction. No differences between groups were detected in the white blood cell count (WBC) (n=3 in each group). Right panel—A representative image of the spleen stained by H&E, shows vast majority of nucleated cells consistent with erythroid precursors in the control group, typical of extramedullary hematopoiesis that occurs in response to anemia. * P<0.05, ** P<0.005. FIG. 2D—Total NO levels were measured by dedicated HPLC system in blood samples and distal colon section, before and after colitis induction. Results show significant elevation in the NO levels after colitis induction in the RBC and colon in the control group but not in the As1^{f/f} Vav Cre^{+/-} mice (n≥3 in each group). FIG. 2E—NO₂ elevation is seen in peritoneal macrophages medium, after LPS (Lipopolysaccharides) administration. The elevation is significantly higher in the control group compare to As1^{f/f} Vav Cre^{+/-} group (n=6 in each group). FIG. 2F—A significant reduction in glucose levels and elevation in lactate levels in response to LPS administration is demonstrated in the control but not in the As1^{f/f} Vav Cre^{+/-} MQs. Measurement performed using NOVA (n≥4 in each group). FIG. 2G—A significant elevation in the citrate shown in the As1^{f/f} Vav cre^{tg/+} peritoneal macrophages after LPS administration, measured by GC (n=3 in each group). FIG. 2H—Immunohistochemistry staining for the general number of macrophages as well as activated macrophages, in distal colon section after colitis induction, shows significant reduction in the number of activated macrophages in the As1^{f/f} Vav Cre^{+/-} mice compare to control. Cells were counted from at least 8 microscopic fields and taken from 3 different animals. Right panel—quantification analysis was performed in a blinded manner. * P<0.05, ** P<0.005.

FIGS. 3A-J demonstrate that NO related metabolites alleviate colitis severity. Treatment with the arginine pathway related metabolites shows significant improvement in the colitis compare to control. Colitis was induced on C57BL/6J.OlaaHsd mice. Mice were treated with 1% DSS for 6 days, followed by administration of either arginine 1% (wt/vol) solution in drinking water, citrulline 1%, NaNO₂ 100 mg, or water as control for additional 6 days. FIG. 3A—A significant weight loss reduction in the control group compared to the three treatments (n≥25 in each group). FIG. 3B—Endoscopic evaluation on day 6 after colitis induction before treatment shows no differences between the groups. Significant improvement in the colitis score on day 12, was observed for all three treatments. In comparison to arginine, citrulline was more beneficial treatment (n≥25 in each group). FIG. 3C—A significant reduction in colon length in the control group as compared to the treated colons (n≥15 in each group). FIG. 3D—Histology score for colitis was significantly lower in the treated colons as compared to the control. FIG. 3E—Immunohistochemistry staining of distal colon sections shows up regulation of As1 expression in the control group following colitis induction. A more pronounce expression of As1 was seen in the group treated with citrulline while the group treated with NaNO₂ showed reduced expression of As1. * P<0.05, ** P<0.005. FIGS. 3F-H—A Tunnel staining showing increased apoptosis in the control intestine as compare to colon taken from mice treated with citrulline. FIGS. 3I-J depict the results of FACS analysis showing that Citrulline treatment is more effective than arginine in inducing NO synthesis in enterocytes. FIGS. 3I-J—FACS analysis showing that Citrulline treatment is more effective than arginine in inducing NO synthesis in enterocytes.

FIGS. 4A-N demonstrate that induction of endogenous NO production by enterocytes alleviates colitis. Colitis was induced on C57BL/6J.OlaaHsd mice. Mice were treated with 1% DSS for 6 days, followed by administration of either fiselin (1 mg in 30 μl DMSO IP, twice, in 3 days interval) citrulline 1% (wt/vol) solution in drinking water, the combination of both treatments or only DMSO as a control (30 μl DMSO IP, twice, in 3 days interval). FIG. 4A—Immunohistochemistry staining of distal colon sections after colitis induction shows up regulation of As1 and Asst expression after fisetin treatment. FIG. 4B—A significant weight loss is documented in the control group compared to the treated mice (n≥20 in each group). FIG. 4C—An endoscopic evaluation before treatment shows no differences between the groups while there is a significant improvement on day 12 in the colitis score in the treated groups as compared to control. Among treatments, the combined treatment was the most beneficial one (n≥20 in each group). FIG. 4D—A significant longer colon length is seen in the treated groups as compared to control. In addition, the colon is significantly longer in the combined treatment as compared to each treatment separately (n=15 in both groups). FIG. 4E—A significant reduction in histological score in all treatment groups as compared to control. FIGS. 4F-G—FACS analysis of colon epithelial cells from mice treated with combined treatment show significant increase in NO levels compared to control group (n=4 in each group). FIGS. 4H-K—FACS analysis of colon cells from mice treated with fisetin versus control after colitis induction shows significant increase in NO levels (FIGS. 4H-I) and iNOS expression (FIGS. 4J-K) in the fisetin treated group. FIGS. 4L-N—Colitis was induced on enterocytes CKO mice and compared to controls. Mice were treated with 0.8% DSS for 5 days, followed by administration with either NO donors; NaNO₂ (100 mg/kg) or fisetin (1 mg in 30 μl DMSO IP, twice, in 3 days interval). Fisetin treatment was effective in the control group but not in the enterocytes CKO mice as shown by the increased survival (FIG. 4L), increased body weight gain (FIG. 4M) and the lower colitis score (FIG. 4B) in control mice as compared to enterocytes CKO mice; *P<0.05, **P<0.005. Note that colitis severity is reduced by activating As1 in enterocytes, by citrulline and fisetin supplementation.

FIGS. 5A-I show generation of As1 CKO mice and a genetic colitis model, and characterization of As1^{flox/flox} Vav cre^{+/-}. FIG. 5A—PCR conducted on blood samples, shows expression of Vav Cre but not As1 in the As1^{f/f} Vav cre^{+/-} mice (n=4 in both groups). FIG. 5B—RT PCR showing decreased As1 expression in As1^{f/f} Vav cre^{+/-} in mice spleen and blood samples (n=3 in both groups). FIG. 5C—A complete blood count shows no differences between the As1^{f/f} Vav cre^{+/-} and control groups, before colitis induction. WBC; white blood cells, RBC; red blood cells, HgB; hemoglobin, HCT; hematocrit. (n=6 in each group) * P<0.05, ** P<0.005. FIGS. 5D-E—A significant reduction in histological score (FIG. 5D) and in colon lengths (FIG. 5E) in the As1^{f/f} Vav cre^{+/-} mice as compared to the control group (n≥15 in each group). FIGS. 5F-G—Colitis was induced on C57BL/6J.OlaaHsd mice with 1% DSS for 6 days, followed by administration of either fiselin (1 mg in 30 μl DMSO IP, twice, in 3 days interval), citrulline 1% (wt/vol) solution in drinking water, the combination of both treatments or only DMSO as a control (30 μl DMSO IP, twice, in 3 days interval). FIGS. 5H-I—A histogram depicting colitis score in mice after 4 weeks (FIG. 5H) or 7 weeks (FIG. 5I) of induction of colitis. Lethally irradiated wild type animals that received a bone marrow (BM) graft from CX₃CR1^{Cre}:I110ra^{fl/fl} developed colitis as indicated by colitis score 4 (FIG. 5H) weeks and 7 weeks (FIG. 5I) after BM implantation. Almost no signs of colitis were observed in animals that received BM from wild type animals* P<0.05, ** P<0.005 A histogram

FIGS. 6A-O demonstrate that intestinal As1 is upregulated at arginine deficient states. FIG. 6A—A Western blot confirming that As1 expression level is decreased in intestine from As1^{f/f} villin cre^{+/-} mice. FIGS. 6B-C—No clinical difference was seen between As1^{f/f} and As1^{f/f} villin cre^{+/-} mice as indicated by body weight change (FIG. 6B) and colitis score (FIG. 6C) on day 7 and 12 after colitis induction (n≥5 in each group). FIG. 6D—While As1 is expressed in young pups, dramatic reduction in observed the As1 expression in the adult colon enterocytes. Supplementing the mice with arginine free diet with and without colitis induction up regulates As1 levels in the As1^{f/f} control mice, as seen by immunohistochemistry staining of the colon for As1. Colitis by itself did not upregulates As1 levels significantly. FIG. 6E—Decreased colon length in the As1^{f/f} Villin Cre^{+/-} group as compared to controls. FIG. 6F—Plasma arginine levels show no differences between the As1^{f/f} Villin cre^{-/+} and control mice fed on normal or Arginine free diet with or without colitis induction (n≥3 in each group). FIGS. 6G-A graph depicting percentage of BW change over time in the As1^{f/f} and As1^{f/f} villin cre^{+/-} mice. FIG. 6H-A histogram depicting the recruitment of immune cells in the As1^{f/f} and As1^{f/f} villin cre^{+/-} mice. FIG. 6I—Colitis score in As1^{f/f} and As1^{f/f} villin cre^{+/-} mice. Arginine deficient diet did not cause growth differences between control and Villin^{Cre}:As1^{f/f} (FIG. 6G) or in immune cell CD45+ recruitment to the intestine as shown by flow cytometry analysis (FACS) (FIG. 6H). Colitis was induced by bone marrow implantation from WT of CX₃CR1^{Cre}:I110ra^{fl/fl} donors, to lethally irradiated Villin^{Cre}:As1^{f/f} and control As1^{fl/fl} mice. While no colitis signs were detected in mice receiving WT BM, Villin^{Cre}:As1^{f/f} mice implanted with CX₃CR1^{Cre}:I110ra^{fl/fl}BM had increased severity of colitis as compared to control mice as demonstrated by a higher endoscopic colitis score (FIG. 6I) (n≥3 in each group); shorter colon length (FIG. 6J) (n≥3 in each group) and higher histologic score due to increased infiltration, edema and ulcers as shown by H&E staining (FIGS. 6K-M). LC/MS measurement of arginine shows significantly reduced levels in the enterocytes of Villin^{Cre}:As1^{f/f} mice as compared to the control mice (FIG. 6N). Polyamine measurements (FIG. 6O) using LC/MS show significantly reduced levels of ornithine and putrescine in enterocytes of Villin^{Cre}:As1^{f/f} mice as compared to the control mice, while no differences were observed in the spermidine and spermine levels.* P<0.05, ** P<0.005, ***P<0.0005, ****P<0.00005.

FIG. 7 is a formula depicting the structure of citrulline.

FIGS. 8A-K show that decreased NO levels in enterocytes increase colitis severity. Analysis of colon cells from Villin^{Cre}:As1^{f/f} and in control As1^{f/f} mice by flow cytometry analysis (FACS) and ELISA, before and after colitis induction by DSS. FIGS. 8A-B—NO expression in CD45-cells shows significant increase in the control group after colitis induction but not in the Villin^{Cre}:As1^{f/f} mice (n=3 in each group). FIG. 8C-D—S-nitrosocystein (marked as “SNO”) expression by CD45-cells—(i.e. non-hematopoietic cells), shows significant increase in nitrosylation in the control group after colitis induction but not in the Villin^{Cre}:As1^{f/f} mice shown by ELISA (FIG. 8C) (n=3 in each group) as well as by FACS analysis (FIG. 8D). FIGS. 8E-F—Significant increase in iNOS expression in CD45-CD326+(i.e. colonic enterocytes) in the control group but not in the Villin^{Cre}:As1^{f/f} after colitis induction. FIGS. 8G-I—Increased immune system cells CD45+ recruitment to the colon in the Villin^{Cre}:As1^{f/f} mice compared to control. FIGS. 8J-K—Similar upregulation of NO and iNOS in CD64+, CD11c+ colon macrophages in Villin^{Cre}:As1^{f/f} mice and the control group.

FIGS. 9A-F show decreased NO levels in As1 immune cells CKO. Colitis was induced by DSS in CD11c^{Cre}:As1^{f/f} and in control As1^{f/f} mice. FIGS. 9A-C—Representative images and a summary showing that CD11c^{Cre}:As1^{f/f} mice (FIG. 9B) have decreased severity of colitis as compared to control mice (FIG. 9A) as demonstrated by endoscopic evaluation on days 12 after colitis induction that shows significant improvement in the colitis score of the CD11c^{Cre}:As1^{f/f} group compared to the control group (n≥14 in each group). FIGS. 9A-B shows representative images of the colon taken by colonoscopy. FIG. 9C—summary of colitis score. FIG. 9D—A representative graph showing significant increase in colon length in the CD11c^{Cre}: As1^{f/f} mice as compared to the control group (n≥10 in each group). FIG. 9E—ELISA measurement of pro inflammatory cytokines levels in colon tissue extracted from mice after sacrifice on day 11 following colitis induction, showing significant elevation in IL-1, IL-2, IL5 and TNFα in the control group compared to CD11c^{Cre}:As1^{f/f} mice. FIG. 9F—Human data taken from GEO data set analysis (GSE57945, ID:200057945) for RNA sequencing taken from 359 people, shows significant upregulation of ASL and iNOS expression in patients suffering from Crohn's disease (marked as “iCD”) and ulcerative colitis (UC), compared to normal (control subjects devoid of Crohn's disease or ulcerative colitis) while there is no significant change in ODC (ornithine decarboxylase) expression. * P<0.05, ** P<0.005.

FIGS. 10A-E show that enterocytes self-regulation of NO levels is the most beneficial. Colitis was induced on C57BL/6J.OlaaHsd mice with 1% DSS for 6 days, followed by administration of either fiselin (1 mg in 30 μl DMSO IP, twice, in 3 days interval) citrulline 1% (wt/vol) solution in drinking water, the combination of both treatments or only DMSO as a control (30 μl DMSO IP, twice, in 3 days interval). FIG. 10A-A graph showing percentage of BW (Body weight) change along time (days) in the different treated mice. FIG. 10B—colitis score is presented in the different treated mice. FIG. 10C—Histology score in the different treated mice. FIG. 10D—colon length in the different treated mice. No colitis signs were observed in animals implanted with WT BM donor while significant reduction in colitis score in treated animals was seen, 6 weeks after CX₃CR1^{Cre}:I110ra^{fl/fl} BM implantation as shown by reduced colitis score, reduced histology score and increased colon length (n≥5 in each group). FIG. 10E—In vivo intestinal permeability assay to assess barrier function was performed using a 4 kDa FITC-labeled dextran, shows significantly decreased permeability in the treated mice.

FIGS. 11A-E. FIG. 11A—Crystal violet staining on CaCO2 human adenocarcinoma cell line. 6 days after medium was supplemented with either citrulline 1%, fisetin 100 μM, combination of both supplements or control, shows decreased proliferation after the combined treatment as compared to control. FIG. 11B—A quantification graph for the staining. **p-Value<0.005. FIGS. 11C-D—colitis associated cancer model was induced using the AOM-DSS model. As1^{f/f} and Villin^{Cre}:As1^{f/f} Mice were injected with Azoxymethan (AOM) intraperitoneally at a dose of 12.5 mg/kg body weight. In addition, mice were treated with 2% DSS in the drinking water for 5 days, then followed by 16 days of regular water. This cycle was repeated twice, significant elevation in tumor score (FIG. 11C) and tumor number (FIG. 11D) was seen in the Villin^{Cre}:As1^{f/f} group compare to control. FIG. 11E—A summary illustration demonstrating the distinct role for NO in each cell type. While NO produced by macrophages is harmful, NO generated by enterocytes is protective. Colitis severity is reduced maximally by metabolically activating As1 in enterocytes using citrulline and fisetin supplementation. * P<0.05, ** P<0.005.

FIGS. 12A-F show that As1 expression levels in the enterocytes does not affect the microbiome. Stool samples of 12 weeks old As1^{f/f} and Villin^{Cre}:As1^{f/f} mice on arginine free diet since weaning, and As1^{f/f} mice on standard diet were collected for 16S rRNA Analyses. No differences between As1^{f/f} and Villin^{Cre}:As1^{f/f} mice on arginine free diet are seen, as showed by 3D weighted and un-weighted Principal coordinates analysis (PCoA) of UniFrac distances of 16S rRNA (FIGS. 12A-B). Quantification of the UniFrac distances of 3D weighted and un-weighted Principal coordinates analysis (PCoA) of 16S rRNA. P<0.05 by 1-way ANOVA with Bonferroni post test (FIGS. 12C-D), the averages of Phylum bacterial relative abundance in stool samples (FIG. 12E) and Volcano plot showing degree of differential bacteria taxa abundance between As1^{f/f} and Villin^{Cre}:As1^{f/f} mice on arginine free diet (FIG. 12F). P value=0.05, FDR corrected (D). n=6 in each group, * P<0.05.

FIGS. 13A-J show that cell specific As1 deficiency correlates with decreased NO production. FACS analysis of colon cells from Vav1^{Cre}: As1^{f/f} and from control As1^{f/f} mice, after colitis induction by DSS shows significant increase in the levels of As1, NO and iNOS in CD45+ colon immune cells of the control group but not in the Vav1^{Cre}: As1^{f/f} mice (FIGS. 13A-C) as well as in the CD64+, CD11c+ macrophages (FIGS. 13D-F). NO differences in these parameters in CD326 colon enterocytes (G-I) (n≥4 in each group) * P<0.05, ** P<0.005. (FIG. 13J) ASL expression correlates with iNOS in colon of patients with and without IBD, analysis of GEO dataset—GSE57945 ID: 200057945, (R=0.27; pV=2.612e-05).

FIGS. 14A-F show that fisetin does not increase NO levels in intestinal immune cells. FACS analysis of colon cells from mice treated with fisetin vs control after colitis induction shows decrease in As1, NO and iNOS levels in CD45+ immune system cells after fisetin treatment (FIGS. 14A-C) as well as in the CD64+, CD11c+ macrophages (FIGS. 14D-F). (n=4 in each group) * P<0.05, ** P<0.005.

### DESCRIPTION OF SPECIFIC EMBODIMENTS OF THE INVENTION

The present invention, in some embodiments thereof, relates to using citrulline and/or with combination of a flavonoid for the manufacture of a medicament for treatment of a subject having an inflammatory bowel disease (IBD) and/or colon cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

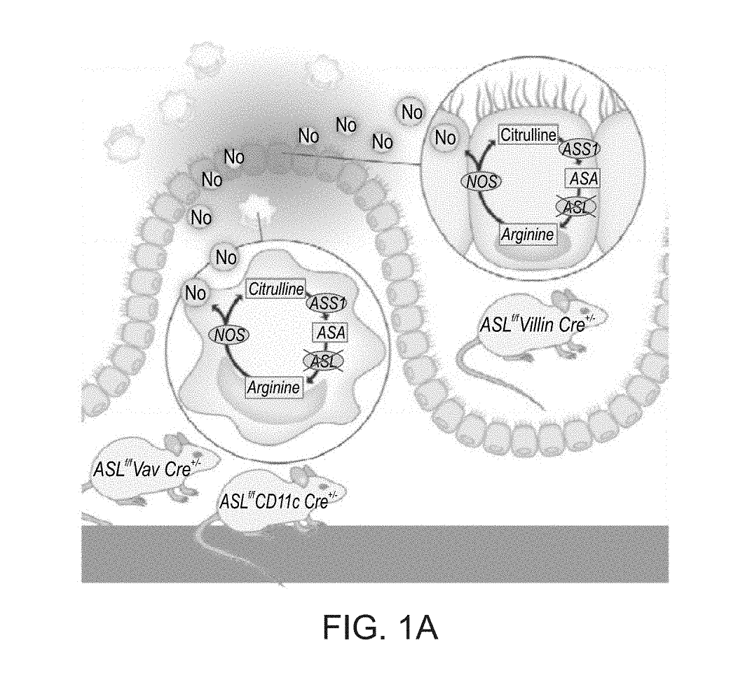

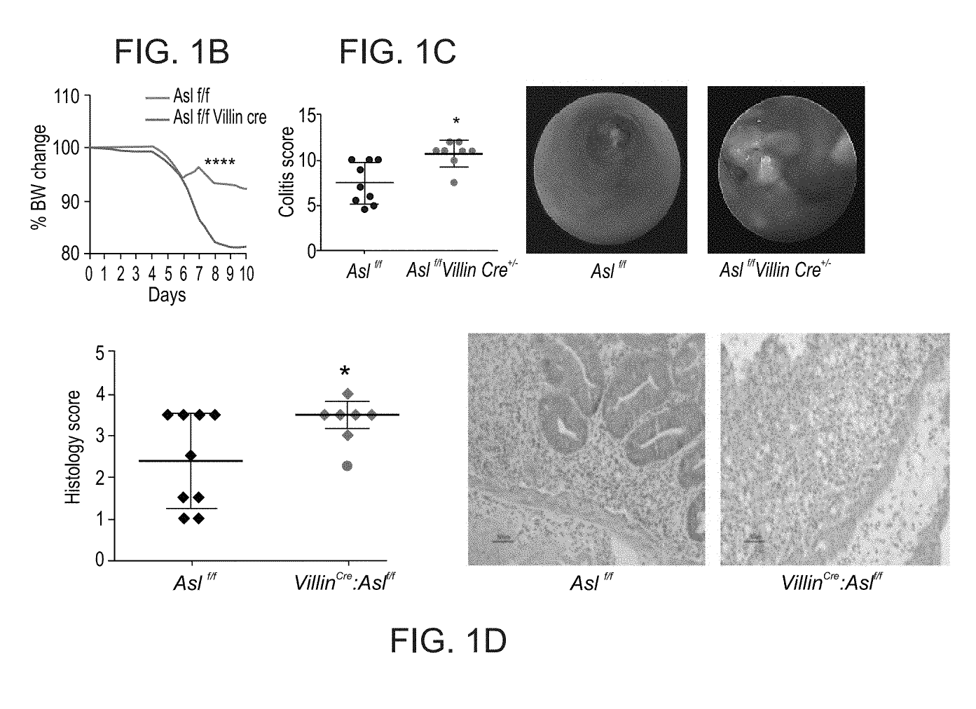

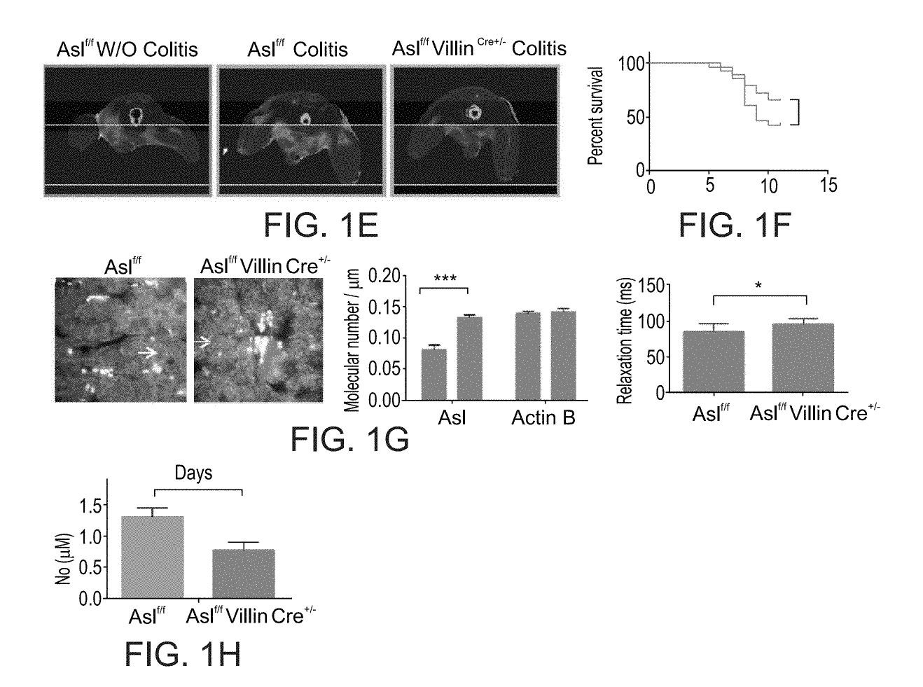

Nitric oxide (NO) is a multifunctional signaling molecule with an established importance for numerous physiological and pathological processes. Yet, for multiple disorders as colitis, there are contradictory data regarding the specific role of NO in disease pathogenesis, preventing NO related drugs implementation into therapy. Argininosuccinate lyase is the only mammalian enzyme able to produce arginine, the substrate for NO generation by all nitric oxide synthase (NOSs). The present inventors decipher the role of NO in IBD by downregulating As1 in enterocytes and in immune cells and characterizing the direct phenotypic and mechanistic consequences of NO metabolic depletion in each cell type, on colitis development and progression with imminent translational implications to therapy for IBD (FIG. 1A). By inducing colitis in different conditional knockout mouse models for As1 deficiency in enterocytes or immune cells, the present inventors find a significantly different sequel for colitis severity; while NO produced by the enterocytes protects from apoptosis, NO generated by the immune cells activates the macrophages and increases colitis severity. iNOS-derived NO from enterocytes alleviates colitis by decreasing macrophage infiltration and tissue damage, whereas immune cell-derived NO is associated with macrophage activation, infiltration and increased severity of inflammation. Thus, the three different adult mouse models of cell specific ASL knockout revealed that ASL expression in enterocytes is essential to generate NO as a defense mechanism to prevent macrophage infiltration, preserve epithelial cell barrier and alleviate the course of colitis. Moreover, synergistic metabolic induction of endogenous NO production by both its substrate and the relevant enzymes, allows the cells to adjust and fine tune NO levels accurately to its needs and is hence the key for successful translation of NO related drugs into therapy. In addition, metabolic induction of endogenous NO production specifically by the enterocyte iNOS, results in improved epithelial integrity and alleviation of colitis and its related complications as colon cancer.

According to an aspect of some embodiments of the invention there is provided a use of a therapeutically effective amount of citrulline for the manufacture of a medicament for treatment of a subject having an inflammatory bowel disease (IBD) and/or colon cancer.

According to an aspect of some embodiments of the invention there is provided a use of a therapeutically effective amount of citrulline and a therapeutically effective amount of a flavonoid for the manufacture of a medicament for treatment of a subject having an inflammatory bowel disease (IBD) and/or colon cancer.

According to an aspect of some embodiments of the invention there is provided a method of treating inflammatory bowel disease (IBD) and/or colon cancer comprising administering to a subject in need thereof a therapeutically effective amount of citrulline, thereby treating the subject.

According to an aspect of some embodiments of the invention there is provided a method of treating inflammatory bowel disease (IBD) and/or colon cancer comprising administering to a subject in need thereof a therapeutically effective amount of citrulline and a therapeutically effective amount of a flavonoid, thereby treating the subject.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology. Preferably, this term encompasses individuals who are at risk to develop the pathology.

As used herein the phrase “inflammatory bowel disease (IBD)” refers to a pathology characterized by an inflammatory condition of the colon and the small intestine, Crohn's disease (CD) and ulcerative colitis (UC) are the principal types of inflammatory bowel disease.

According to some embodiments of the invention, the IBD is not associated with an altered motility of the intestinal tract.

According to an alternative or an additional embodiment the IBD is not a pyloric stenosis disorder.

According to some embodiments of the invention, the IBD comprises ulcerative colitis.

As used herein the phrase “ulcerative colitis (UC)” refers to a chronic relapsing form of inflammatory bowel disease (IBD) that causes inflammation and ulcers in the colon (either in segments or completely) and rectum. Symptoms can range from mild to severe with disease onset usually occurring in young adults. The hallmark symptom of active disease is diarrhea mixed with blood. UC is an intermittent disease, with periods of exacerbated symptoms alternating with relatively symptom-free periods. Although the symptoms of UC can sometimes diminish on their own, the disease usually requires treatment to go into remission.

According to some embodiments of the invention, the IBD comprises Crohn's disease.

As used herein the phrase “Crohn's disease” refers to a type of inflammatory bowel disease (IBD) that may affect any part of the gastrointestinal tract from mouth to anus. Signs and symptoms often include abdominal pain, diarrhea (which may be bloody if inflammation is severe), fever, and weight loss. Other complications may occur outside the gastrointestinal tract and include anemia, skin rashes, arthritis, inflammation of the eye, and feeling tired. The skin rashes may be due to infections as well as pyoderma gangrenosum or erythema nodosum. Bowel obstruction also commonly occurs and those with the disease are at greater risk of bowel cancer.

According to some embodiments of the invention, treating the IBD is manifested by a mucosal healing.

The effect of treatment with the agent(s) according to some embodiments of the invention can be evaluated using known and accepted medical indexes and/or calculators.

For example, a suitable scoring system for ulcerative colitis is the Mayo score (Rutgeerts P, Sandborn W J, Feagan B G, Reinisch W, et al. Infliximab for induction and maintenance therapy for ulcerative colitis. N Engl J Med. 2005 Dec. 8; 353(23):2462-76). The Mayo score ranges from 0 to 12, with higher scores indicating more severe disease. This score can be used for both initial evaluation and monitoring treatment response.

TABLE 1
Mayo Scoring System for Assessment
of Ulcerative Colitis Activity

| | |
|---|---|
| | Stool Frequency (*) |
| 0 | Normal number of stools for patient |
| 1 | 1 to 2 stools per day more than normal |
| 2 | 3 to 4 stools more than normal |
| 3 | >=5 stools more than normal |
| | Rectal Bleeding (**) |
| 0 | No blood seen. |
| 1 | Streaks of blood with stool less than half the time. |
| 2 | Obvious blood with stool most of the time. |
| 3 | Blood alone passes. |
| | Endoscopic findings |
| 0 | Normal or inactive disease. |
| 1 | Mild Disease (erythema, decreased vascular pattern, mild friability) |
| 2 | Moderate Disease (Marked erythema, lack of vascular pattern, |
| | friability, erosions) |
| 3 | Severe Disease (spontaneous bleeding, ulceration) |
| | Physician's Global Assessment (***) |
| 0 | Normal |
| 1 | Mild disease |
| 2 | Moderate disease |
| 3 | Severe disease |
| | Total Points: 2-5 (mild disease); 6-12 (moderate-severe disease) |

Table 1:
(*) Each patient serves as his own control to establish the degree of abnormality of the stool frequency;
(**) The daily bleeding score represents the most severe bleeding of the day.
(***) The physician's global assessment acknowledges the tree other criteria, the patient's daily recollection of abdominal discomfort and general sense of well being, and other observations such as physical findings and the patient's performance status.

Parameters which indicate a positive effect of the treatment of Crohn's disease include, for example, reduction in the number of liquid or very soft stools; reduction in the abdominal pain; reduction in symptoms or findings presumed related to Crohn's disease:arthritis or arthralgia, iritis or uveitis, erythema nodosum, pyoderma gangrenosum, aphthous stomatitis, anal fissure, fistula or perirectal abscess, other bowel-related fistula, febrile (fever), episode over 100 degrees during past week; and/or reduction in abdominal mass.

For example, to assess the therapeutic effect of the method of treating Crohn's disease the Crohn's Disease Activity Index (CDAI) can be used. This calculator gauges the progress or lack of progress for people with Crohn's disease. It is accepted that CDAI scores below 150 indicate a better prognosis than higher scores.

The CDAI calculator takes into consideration the following parameters:

(1). Number of liquid or very soft stools in one week;
(2). Sum of seven daily abdominal pain ratings: (0=none, 1=mild, 2=moderate, 3=severe);
(3). Sum of seven daily ratings of general well-being: (0=well, 1=slightly below par, 2=poor, 3=very poor, 4=terrible)
(4). Symptoms or findings presumed related to Crohn's disease: arthritis or arthralgia, iritis or uveitis, erythema nodosum, pyoderma gangrenosum, aphthous stomatitis, anal fissure, fistula or perirectal abscess, other bowel-related fistula, febrile (fever) episode over 100 degrees during past week;
(5). Taking Lomotil or opiates for diarrhea;
(6). Abnormal mass: 0=none; 0.4=questionable; 1=present
(7). Hematocrit [(Typical−Current)×6] Normal average: For Male=47 For Female=42;
(8). 100×[(standard weight-actual body weight)/standard weight]

Additionally or alternatively, the clinical status of patients with CD following treatment with the agent(s) of some embodiments of the invention can be evaluated using the Harvey-Bradshaw index (HBI) which was devised in 1980 as a simpler version of the Crohn's disease activity index (CDAI) for data collection purposes. It consists of only clinical parameters.

Following is a non-limiting an exemplary calculator for score using the HBI index.

TABLE 2
Table 2: Harvey-Bradshaw index (HBI).

| Parameter | Scoring |
|---|---|
| General well-being | very well +0 |
| | slightly below par +1 |
| | poor +2 |
| | very poor +3 |
| | terrible +4 |
| Abdominal pain | none +0 |
| | mild +1 |
| | moderate +2 |
| | severe +3 |
| Number of liquid stools per day | |
| Abdominal mass | none +0 |
| | dubious +1 |
| | definite +2 |
| | definite and tender +3 |
| Complications | none +0 |
| | arthralgia +1 |
| | uveitis +1 |
| | erythema nodosum +1 |
| | aphthous ulcers +1 |
| | pyoderma gangrenosum +1 |
| | anal fissure +1 |
| | new fistula +1 |
| | abscess +1 |

Patients with Crohn's disease who scored 3 or less on the HBI are very likely to be in remission according to the CDAI. Patients with a score of 8 to 9 or higher are considered to have severe disease.

According to some embodiments of the invention, the disease is colon cancer and/or “colorectal cancer” which are interchangeably used herein.

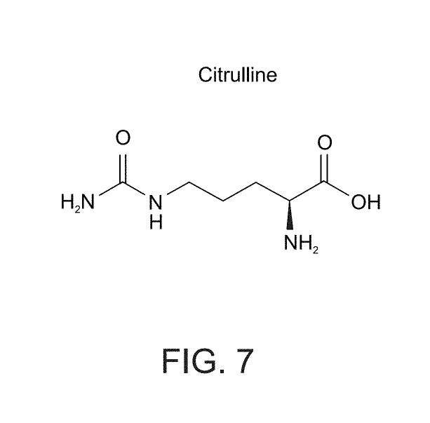

As used herein the term “citrulline” refers to naturally-occurring citrulline or a functional analogue thereof. The naturally-occurring citrulline is a compound having the following formula: H₂NC(O)NH(CH₂)₃CH(NH₂)CO₂H) as depicted in FIG. 7 (also known as 2-Amino-5-(carbamoylamino)pentanoic acid). In the body, citrulline is produced as a byproduct of the enzymatic production of nitric oxide from the amino acid arginine, catalyzed by nitric oxide synthase (Shown schematically in FIG. 1A).

The phrase “citrulline analogue” refers to a compound of formula I, its D,L racemic mixture, its L-isomers and salts thereof like for instance but not limited to salts of inorganic or organic acids like oxalic acid, lactic acid, citric acid, fumaric acid, acetic acid, phosphonic acid, HCl, HBr, sulfuric acid, p-toluol-sulfonic acid. Also suitable are salts of bases such as sodium-, potassium-, or calcium hydroxide, of ammonia, or of amines like ethanolamine, diethanolamine, tri-ethanolamine, N-methylglucamin, tris-(hydroxymethyl)-methylamine or bis-cyclohexylamine just to name a few.

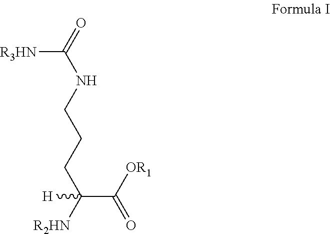

wherein

R₁ has the meaning of hydrogen, alkyl, alkenyl, aryl, phenacyl, omega-hydroxyalkyl or omega-methoxyalkyl,

R₂ and R₃ may be selected independently from hydrogen, alkyl, aryl, acetyl, benzoyl, tert.-butoxycarbonyl, wherein alkyl means a branched or linear chain with 1 to 10 carbons;

alkenyl means a branched or linear chain with 1 to 10 carbons containing up to 3 double bonds;

aryl means a phenyl or naphthyl moiety, optionally substituted once or twice with methyl, nitro, amino or chlorine,

benzoyl may be optionally substituted once or twice with methyl, nitro, amino or chlorine,

phenacyl means a group of formula —(CH₂n(C═O)aryl, and

n can be 1 to 3.

The compounds are commercially available or can be synthesized from citrulline or from ornithine with known methods. The salts can be bought or are easily available by stirring the amino acid with another acid or base in solvents, such as, e.g., ethanol or dioxane or dialkylether or tetrahydrofuran. The following compounds may be taken as examples:
D,L-citrulline,
L-citrulline,
L-citrulline, monoacetate
L-citrulline, hydrochloride
L-citrulline methylester,
L-citrulline ethylester,
L-citrulline-n-hexylester,
L-citrulline (benzoylmethyl)ester, alpha-N-benzoyl-L-citrulline methylester,
N-Boc-L-citrulline,
N1-2,4-dinitrophenyl-D,L-citrulline.

According to some embodiments of the invention, the circulline or the circulline analogues can be provided to the subject in a dose per day in a range of between about 200 mg to about 10 grams, e.g., between about 500 mg to about 10 grams, e.g., between about 800 mg to about 10 grams, e.g., between about 1000 mg to about 10 grams, e.g., between about 1200 mg to about 10 grams, e.g., between about 1400 mg to about 10 grams, e.g., between about 1500 mg to about 10 grams, e.g., between about 2000 mg to about 10 grams, e.g., between about 2500 mg to about 10 grams, e.g., between about 3000 mg to about 10 grams, e.g., between about 3500 mg to about 10 grams, e.g., between about 4000 mg to about 10 grams, e.g., between about 4500 mg to about 10 grams, e.g., between about 5000 mg to about 10 grams, e.g., between about 6-10 grams, e.g., between about 7-10 grams, e.g., between about 8-10 grams, e.g., about 9 grams.

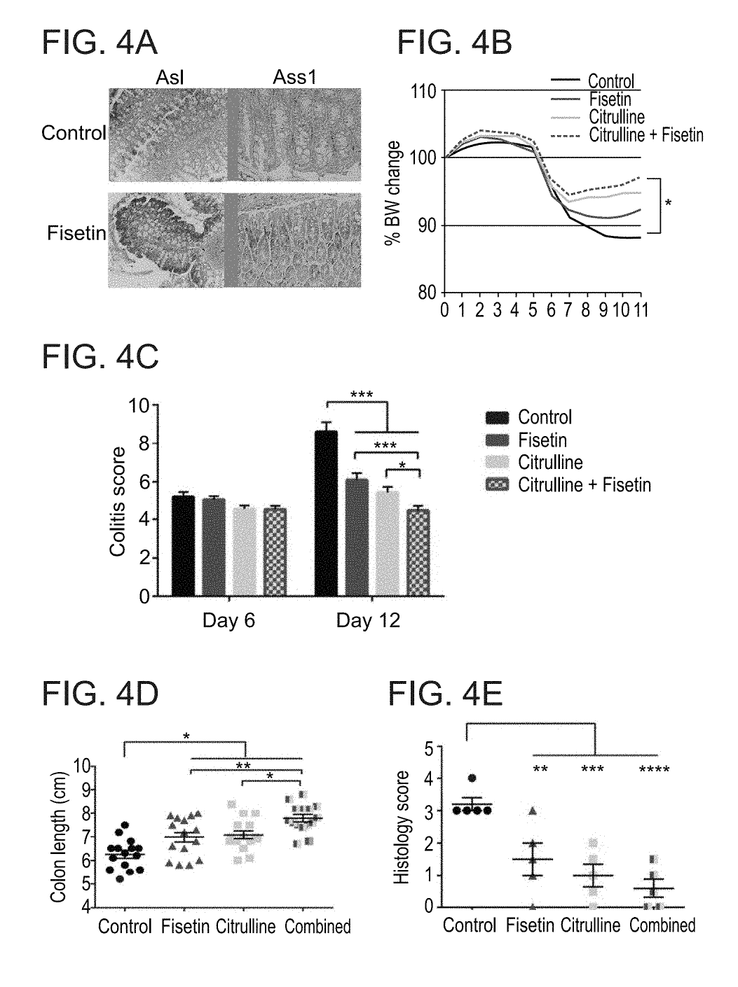

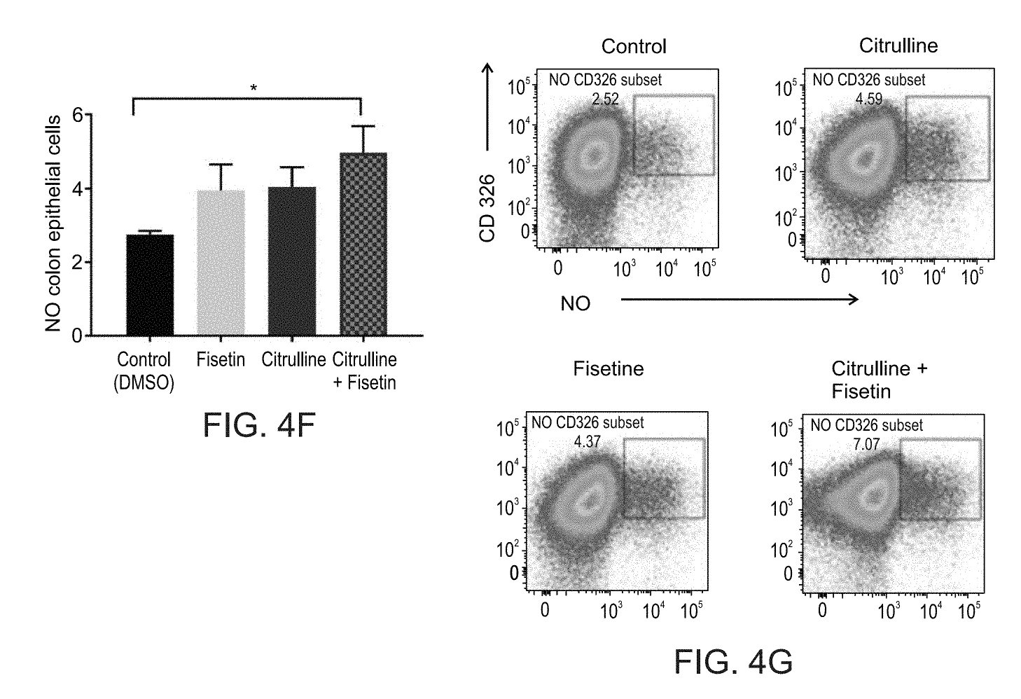

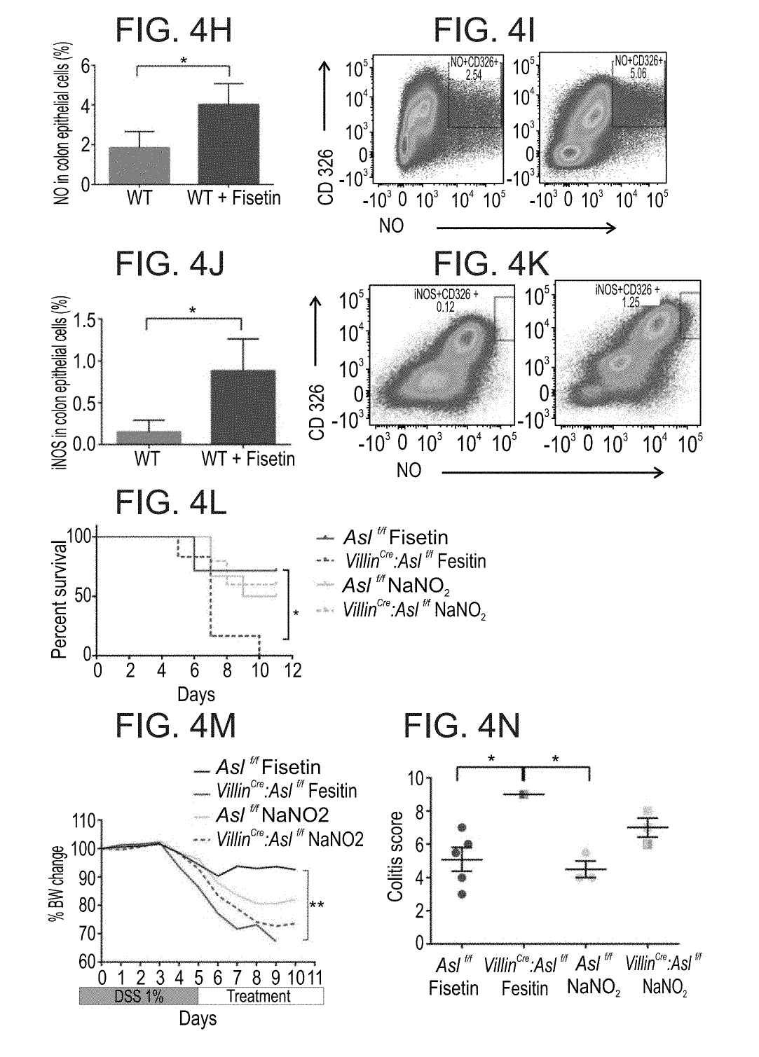

As shown in FIGS. 4A-F the combined treatment of mice having colitis with citrulline and a flavonoid such as fisetin was significantly more effective than treating with any of the agents alone.

According to some embodiments of the invention, the citrulline is co-administered with a therapeutically effective amount of a flavonoid.

As used herein the term “flavonoid” refers to a secondary metabolite having a chemical structure of a 15-carbon skeleton, which consists of two phenyl rings and heterocyclic ring. This carbon structure can be abbreviated C6-C3-C6. The flavonoids can be classified into: (i) flavonoids or bioflavonoids; (ii) isoflavonoids, derived from 3-phenylchromen-4-one (3-phenyl-1,4-benzopyrone) structure; and (in) neoflavonoids, derived from 4-phenylcoumarine (4-phenyl-1,2-benzopyrone) structure. These three flavonoid classes are all ketone-containing compounds, and as such, are anthoxanthins.

Example of flavonoids which can be used according to some embodiments include, but are not limited to flavones (2-phenylchromen-4-one, Luteolin, Apigenin, and Tangeritin) and flavonols (3-hydroxy-2-phenylchromen-4-one, e.g., Quercetin, Kaempferol, Myricetin, Fisetin, Galangin, Isorhamnetin, Pachypodol, Rhamnazin, Pyranoflavonols, and Furanoflavonols); Flavanones (2,3-dihydro-2-phenylchromen-4-one, e.g., Hesperetin, Naringenin, Eriodictyol, and Homoeriodictyol); flavanonols (or 3-Hydroxyflavanone, or 2,3-dihydroflavonol (3-hydroxy-2,3-dihydro-2-phenylchromen-4-one), e.g., Taxifolin (or Dihydroquercetin), Dihydrokaempferol); Flavan structure such as flavan-3-ols (flavanols), flavan-4-ols and flavan-3,4-diols (e.g., (leucoanthocyanidin)); Catechin (C), Ciallocatechin (GC), Catechin 3-gallate (Cg), Gallocatechin 3-gallate (GCg), Epicatechins (Epicatechin (EC)), Epigallocatechin (EGC), Epicatechin 3-gallate (ECg), Epigallocatechin 3-gallate (EGCg); Theaflavin Theaflavin-3-gallate, Theaflavin-3′-gallate, Theaflavin-3,3r-digallate); Thearubigin; Proanthocyanidins are dimers, trimers, oligomers, or polymers of the flavanols; Anthocyanidins (aglycones of anthocyanins; they use the flavylium (2-phenylchromenylium) ion skeleton, Examples: Cyanidin, Delphinidin, Malvidin, Pelargonidin, Peonidin, Petunidin); Flavylium skeleton of anthocyanidins; isoflavonoids

[Isoflavones use the 3-phenylchromen-4-one skeleton (with no hydroxyl group substitution on carbon at position 2)] e.g., Genistein. Daidzein, Glycitein; Isoflavones; Isoflavandiols; Isoflavenes; Coumestans; Pterocarpans.

According to some embodiments of the invention, the flavonoid is a small molecule.

According to some embodiments of the invention, the flavonoid is naturally-occurring flavonoid.

According to some embodiments of the invention, the flavonoid is fisetin. According to some embodiments of the invention, the naturally-occurring flavonoid is fisetin.

Fisetin (3, 7, 3′, 4′-tetrahydroxyflavone) is a flavonol. It can be found in many plants, where it serves as a colouring agent. It is also found in many fruits and vegetables, such as strawberries, apples, persimmons, onions and cucumbers.

According to some embodiments of the invention, the therapeutically effective amount of the citrulline and/or of the flavonoid (e.g., fisetin) upregulates the level of nitric oxide in enterocyte cells of the subject.

Enterocytes (intestinal absorptive cells) are simple columnar epithelial cells found in the small intestine, which secret water and electrolytes.

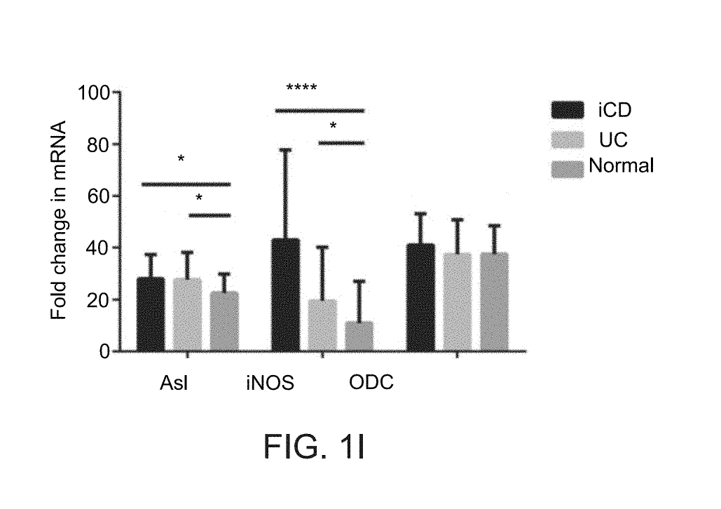

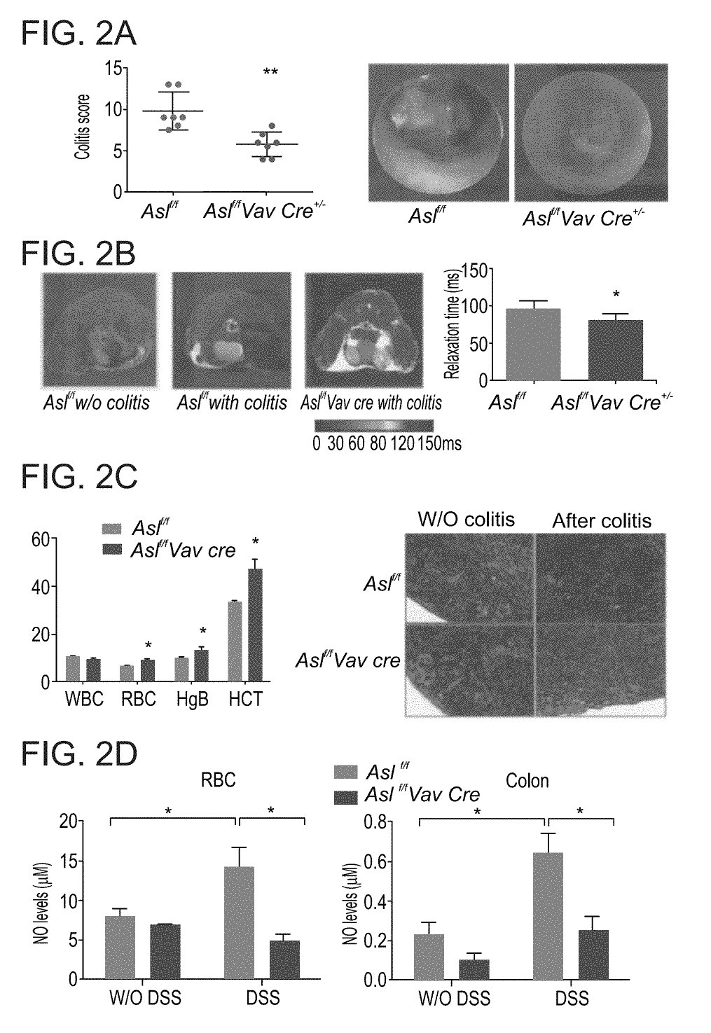

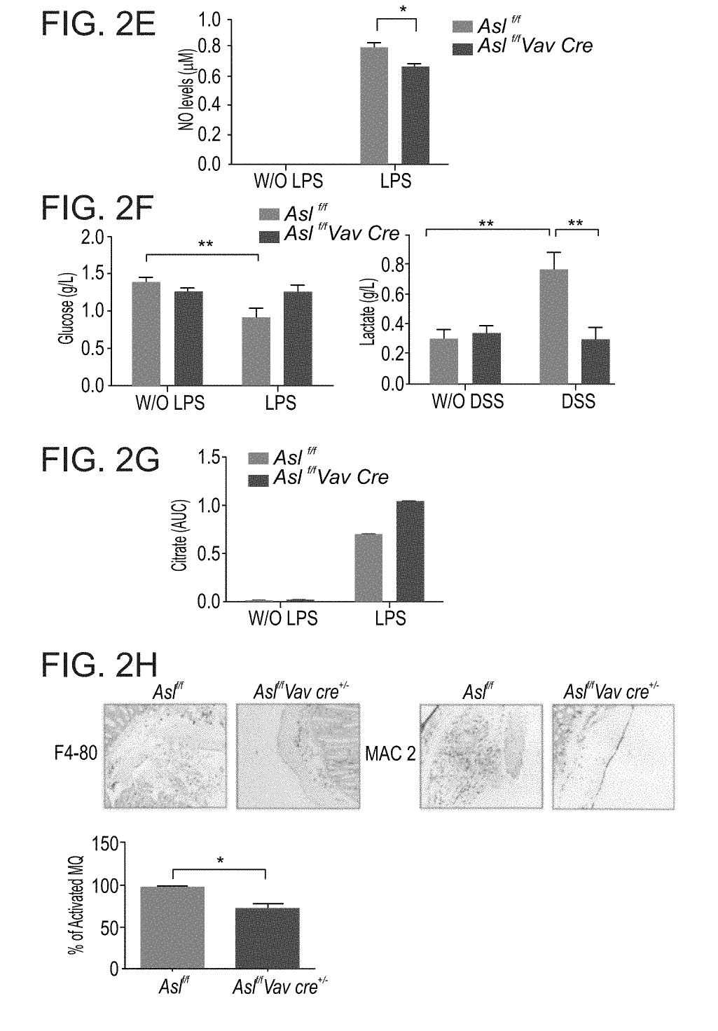

Assays which can be used to determine the level of nitric oxide (NO) in the enterocyte cells of the subject include using an HPLC system for measuring NO in a colon tissue biopsy obtained from the subject. In general NO₂ and NO₃ are accepted bio markers of NO. Exemplary assays are shown in FIGS. 1H, 2D (right panel, marked “colon”). Generally, when both biomarkers exhibit an effect then the effect can be summed for both and is marked “NO”; if only one biomarker (i.e., NO₂ or NO₃) exhibits an effect, then the biomarker that exhibited the effect is mentioned.

According to some embodiments of the invention, the therapeutically effective amount of the citrulline and/or of the flavonoid (e.g., fisetin) upregulates the level of Argininosuccinate Lyase (ASL) in the enterocyte cells.

As used herein the phrase Argininosuccinate Lyase (ASL) refers to the enzyme which forms a cytosolic homotetramer and primarily catalyzes the reversible hydrolytic cleavage of argininosuccinate into arginine and fumarate, an essential step in the liver in detoxifying ammonia via the urea cycle. There are several isoforms of this enzyme resulting from alternative splicing: isoform 1, the longest isoform (GenBank Accession No. NP_000039.2; SEQ ID NO:1), encoded by two variants, variant (1) (GenBank Accession No. NM_001024943.1; SEQ ID NO:2) and variant (2) (GenBank Accession No. NM_000048.3; SEQ ID NO:3); isoform 2 (GenBank Accession No. NP_001020115.1; SEQ ID NO:4; encoded by GenBank Accession No. NM_001024944.1; SEQ ID NO:5) and isoform 3 (GenBank Accession No. NP_001020117.1; SEQ ID NO:6) encoded by GenBank Accession No. NM_001024946.1; SEQ ID NO:7).

Assays which can be used to determine the level of Argininosuccinate Lyase (ASL) in the enterocyte cells of the subject include any protein detection method suitable for a tissue biopsy, e.g., as obtained from a colon of the subject. The protein detection method can employ an antibody specifically recognizing the human Argininosuccinate Lyase (ASL) in a colon or other parts of the gastrointestinal tissue of the subject. The protein detection methods can be for example, immunohistochemistry, Western blot analysis, immunofluorescence, radio immuno assay and the like.

Exemplary antibodies which specifically recognize ASL and which can be used include, but are not limited to Anti-Argininosuccinate Lyase antibody such as EPR19396 (ab201026); ab97370; [3D4] (ab180361); [EPR19382] (ab201025); ab199117; ab96215; ab67512; ab128631; Available from various manufacturers such as abcam, Cambridge, Mass. USA.

Thus, the level of NO and/or of ASL in the enterozytes of the subject can be determined following treatment with the agent(s) of the invention in order to monitor therapeutic effect. According to some embodiment of the invention, the level of NO and/or of ASL in the enterocytes can be determined prior to and following administration of the therapeutic agent(s) into the subject and the changes in these levels can be monitored to determine the therapeutic effect.

According to some embodiments of the invention, the method further comprising administering to the subject an agent which downregulates Argininosuccinate Lyase (ASL) in immune cells of the subject.

According to some embodiments of the invention, the immune cells comprise macrophages.

The citrulline and/or the flavonoid(s) of some embodiments of the invention can be administered to an organism per se, or in a pharmaceutical composition where it is mixed with suitable carriers or excipients.

According to an aspect of some embodiments of the invention there is provided a pharmaceutical composition comprising a therapeutic effective amount of citrulline and a therapeutically effective amount of a flavonoid for the treatment of an inflammatory bowel disease (IBD) and/or colon cancer.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the citrulline and/or the flavonoid(s) of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients [the citrulline and/or the flavonoid(s) of some embodiments of the invention] effective to prevent, alleviate or ameliorate symptoms of a disorder (e.g., IBD and/or colon cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels (e.g., level within the enterocytes) of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention there is provided a kit for the treatment of an inflammatory bowel disease (IBD) and/or colon cancer comprising a first container packaging a first pharmaceutical composition comprising a therapeutic effective amount of citrulline, and a second container packaging a second pharmaceutical composition comprising therapeutically effective amount of a flavonoid.

It should be noted that since citrulline can be dissolved in water, the carrier or a diluent used in the first pharmaceutical composition which comprises citrulline can be water-based.

It should be noted that since fisetin can be dissolved in a hydrophobic solution (e.g., oil), the carrier or a diluent used in the second pharmaceutical composition which comprises fisetin can be oil-based.

According to some embodiments of the invention, the active agents described hereinabove (e.g., the citrulline and/or the flavonoid of some embodiments of the invention) can be provided to the subject in need thereof along with an additional medicament identified for treating the IBD, i.e., by combination therapy.

For example, a therapeutic regimen for treatment of IBD suitable for combination with the citrulline and/or the flavonoid of some embodiments of the invention include, but are not limited to steroid(s), anti-inflammatory or immunosuppressant drugs.

According to some embodiments of the invention, the active agents described hereinabove (e.g., the citrulline and/or the flavonoid of some embodiments of the invention) can be provided to the subject in need thereof along with an additional medicament identified for treating the colon cancer, i.e., by combination therapy.

Therapeutic regimen for treatment of colon cancer suitable for combination with the citrulline and/or the flavonoid of some embodiments of the invention include, but are not limited to chemotherapy, biological therapy, immunological therapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments of the invention, the method further comprising treating a subject having the colon cancer by a treatment selected from the group consisting of: chemotherapy, biological therapy, radiotherapy, phototherapy, photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments of the invention, the composition further comprises an agent suitable for a treatment selected from the group consisting of: chemotherapy, biological therapy, photodynamic therapy, nutritional therapy, brachiotherapy, immunotherapy, and cellular therapy.

It should be noted that such synergistic activity of treatment with the citrulline and/or the flavonoid of some embodiments of the invention and with the additional therapeutic methods or compositions has the potential to significantly reduce the effective clinical doses of such treatments, thereby reducing the often devastating negative side effects and high cost of the treatment.

Anti-cancer drugs that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc.

### (Health Professions Division).

Approved chemotherapy include, but are not limited to, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

Anti-cancer biological drugs that can be co-administered with the compounds of the invention include, but are not limited to bevacizumab (AVASTIN™ Genentech Inc.), Cetuximab (ERBITUX™ ImClone Systems Incorporated), Panitumumab (VECTIBIX™ Immunex Corporation) and/or any combination thereof.

Additional anti-cancer drugs that can be co-administered with the compounds of the invention include, but are not limited to 5-FU, Capecitabine (XELODA™ Hoffmann-La Roche, Inc), Irinotecan (CAMPTOSAR™ YAKULT HONSHA COMPANY, LTD), Oxaliplatin (ELOXATIN™ Sanofi), Trifluridine and tipiracil (LONSURF™ TAIHO PHARMACEUTICAL CO., LTD.), Gemcitabine (GEMZAR™ Eli Lilly and Company), Albumin-bound paclitaxel (ABRAXANE™ of ABRAXIS BIOSCIENCE, LLC), Cisplatin, Paclitaxel (TAXOL™ Bristol-Myers Squibb Company), Docetaxel (TAXOTERE™ AVENTIS PHARMA S.A.), Irinotecan liposome (ONIVYDE™ Merrimack Pharmaceuticals, Inc.), dacarbazine (DTIC-DOME™ BAYER HEALTHCARE PHARMACEUTICALS INC.), ETOPOSIDE (ETOPOPHOS™ Bristol-Myers Squibb Company), Temozolomide (TEMODAL™ Schering Corporation), lapatinib (Tyverb™ GlaxoSmithKline), erlotinib (Tarceva™ Astellas Pharma Inc.), everolimus (AFINITOR™ Novartis AG), and/or any combination thereof. It should be noted that any combination of known anti-cancer treatment (e.g., biological, immunological, chemotherapy and the like) can be combined with the gene therapy approach of the claimed compositions

Analysis and Discussion

Inflammatory bowel disease (IBD) is comprised of two major disorders: ulcerative colitis (UC) and Crohn's disease (CD). These disorders are chronic inflammatory conditions with episodes of exacerbation and typically require long-term treatment with corticosteroids, cyclosporine, biologic agents such as anti-TNF alpha or anti integrin antibodies, or surgery. The disease burden is significant not only on affected individuals and their families but also on the health care costs. Adjuvant therapy that specifically targets key pathogenic mechanisms would thus be of significant value.

Since the discovery of NO more than 30 years ago, its importance to multiple physiological and pathological processes has been well documented in more than 100,000 studies in pubmed¹⁸. NO has been known to have an important role in the inflammation associated with IBD. Specifically in IBD, a disturbed balance between the proinflammatory and immunoregulatory function of NO in the immune cells and enterocytes, respectively, made it challenging to regulate NO levels from a therapeutic standpoint. However, the cell specific contribution of NO to the body pool has not been deciphered, partly because of the three NOS's isoforms that are able to compensate and mask each other. In addition, the half-life of NO is less than two milliseconds and its donors have non consistent affects, making it is very difficult to regulate NO levels for therapeutics¹⁹. These reasons together defer implementation of NO related drugs into therapy in spite of its un-doubtful importance.

Since all three isoforms require arginine and ASL in order to produce NO⁶, the present inventors aimed to abolish ASL expression in a cell-specific manner, to metabolically decrease NO generation in a cell specific manner, and to dissect the cell specific role of NO in IBD pathogenesis. As a proof of concept for the differences in the role of NO in different cell types, the present inventors chose to focus on IBD colitis in which both enterocytes and immune cells are the major participants in disease pathogenesis. Indeed the present inventors found a distinct role for NO in each cell type and while NO produced by the MQ induced M1 MQ and cytokine secretion and was hence harmful, NO generated by the enterocytes improved epithelial integrity and was protective.

Thus, the present inventors supplemented NO related drugs in the mouse drinking water and found it to be beneficial in alleviating colitis severity. Furthermore, the present inventors found that increasing ASL and ASS1 expression in the intestine by fisetin, together with providing citrulline as substrate for NO pathway enhancement, achieved the most beneficial outcome. Thus, supplementing the gut with metabolic ability to increase NO generation in the colon, allows it to adjust the level and localization of NO at the exact amount and place where it is needed.

In addition to the positive effect fisetin has been shown to have in colitis in decreasing MQ activity, its combination with citrulline achieved the most beneficial outcome by boosting the metabolic ability of the enterocytes to synthesize and adjust the level and localization of NO to the exact amount and place where it is needed to strengthen the epithelial barrier, preventing immune cells infiltration as a protective mechanism against colitis. Importantly, the increased colitis severity following As1 knockout in enterocytes translates to increased colon cancer tumor burden while supplementing the combined treatment decreases it.

Without being bound by any theory, this study serves as a proof of concept as it conveys three main ideas; first it confirms that regulating ASL allows for metabolic regulation of NO in a cell specific manner. Secondly, it shows that during disease, cell-autonomous production of NO by iNOS can be protective as the present inventors show here for enterocytes during colitis. Lastly and most importantly, this study demonstrates the superior metabolic advantage of supplementing with the substrate and the ability to upregulate the relevant metabolic enzymes required for the synthesis of specific metabolite, over giving the deficient molecule directly as therapy. Since both citrulline and fisetin are neutraceutical agents that are available as supplements, the findings from this study could be translated rather quickly in the human context.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 2 is expressed in a DNA sequence format (e.g., reciting T for thymine), but it can refer to either a DNA sequence that corresponds to an Argininosuccinate Lyase nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture”

Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Animals:

Animal experiments were approved by the Weizmann Institute Animal Care and Use Committee Following US National Institute of Health, European Commission and the Israeli guidelines. (IACUC: 07291113-2, 08020114-2, 24330116-3, 15990115-1, 24340116-2). Mice were purchased from Jackson Laboratory, Bar Harbor, Me., USA; B6.Cg-Tg(Vav1-icre)A2 Kio/J and B6.SJL-Tg(Vil-cre0997 Gum/J.

Colitis Induction:

Colitis was induced in Vav1^{Cre}:As1^{f/f} mice and CD11c^{Cre}:As1^{f/f} with 1.5% DSS and with 0.8% DSS in the Villin^{Cre}:As1^{f/f} (wt/vol), (Dextran Sodium Sulfate, MP Biomedicals; molecular weight 36,000-50,000 Da). The powder was dissolved in the drinking water for 6 days, followed by 5 days of supplementing regular water²⁰. Each group consisted of eight to ten, 8-10 weeks old, male mice. Experiments were repeated at least three times. “f/f”—flox/flox.

Arginine Free Diet:

mice received arginine free diet (Envigo, TD09152 TEKLAD) since weaning and along the experiment.

Endoscopic Evaluation of Colitis:

Colonoscopy was performed on days 7 and 11 of the experiment to monitor for severity of colitis. For the procedure, a high resolution mouse video endoscopic system was used, which consists of a miniature endoscope (scope 1.9 mm outer diameter), a xenon light source, a triple chip camera, and an air pump to achieve regulated inflation of the mouse bowel (Karl Storz, Tuttlingen, Germany). Mice were anesthetized by the administration of 4-5% of isoflurane in 100% oxygen at a rate of 0.2-0.5 L/min or by the administration of 100 mg/kg Ketamine/10 mg/kg Xylazine mixture IP, after which, the endoscope was introduced via the anus and the colon was carefully insufflated with an air pump before analysis of the colonic mucosa. The endoscopic procedure was viewed on a color monitor and digitally recorded on tape. Colitis was scored according to the Murine endoscopic index of colitis severity (MEICS), considering differences in the thickness of the bowel wall, changes in blood vessel integrity, mucosal surface, stool consistency or the presence of fibrin, scored each between 0 and 3. The cumulative score ranged from 0 (no signs of inflammation) to 15 (signs of severe inflammation)²¹.

Drug Administration:

Mice were treated with 1% (wt/vol) DSS for 6 days, followed by administration of either arginine 1% (wt/vol) solution (Sigma-Aldrich, St. Louis, Mo., catalogue number A8094) in drinking water, citrulline 1% (wt/vol) (L-citrulline, Chem-Impex International), NaNO₂ 100 mg (Sigma-Aldrich, St. Louis, Mo., catalogue number S2252), or water as control for additional 6 days.

Fisetin was given by intraperitoneal (i.p.) injection (1 mg/animal) in 30 μL of DMSO twice weekly. The control group was injected with 30 μL of DMSO twice weekly. Each group consisted of ten, 8-weeks old, male mice. Experiments were repeated three times.

Histopathology. On the day of sacrifice, colons were removed and their length was measured. Colon was fixed in 2.5% PFA solution overnight at 4° C., then embedded in paraffin, sectioned and stained with H&E. Tissues were examined in a blinded manner by a pathologist and scored on a 0-4 scale based on the parameters of inflammation severity²⁰.

Histologic evaluation of CX₃CR1^{Cre}:I110ra^{fl/fl} mice as previously described (E. Zigmond, et al. 2014. “Macrophage-restricted interleukin-10 receptor deficiency, but not IL-10 deficiency, causes severe spontaneous colitis”. Immunity 40, 720-733). Briefly, three segments of the colon (proximal colon, medial colon and distal colon-rectum) were given a score between 0-4 and the summation of these scores provided a total colonic disease score.

Immunohistochemistry:

Four micrometer paraffin embedded tissue sections were deparaffinized and rehydrated. Endogenous peroxidase was blocked with three percent H₂O₂ in methanol. Sections undergoing for ALS staining were incubated in cold acetone at −20° C. for 7 minutes. For F4/80 and MAC-2 staining, the present inventors performed antigen retrieval in Tris-EDTA pH 9 and 10 mM citric acid pH 6, for 10 minutes, respectively, using a low boiling program in the microwave to break protein cross-links and unmask antigens. After pre-incubation with 20% normal horse serum and 0.2% Triton X-100 for 1 hour at RT (root temperature), sections were incubated with the primary antibodies as follow; ASL (1:100, Abcam, ab97370, CA, USA); F4/80 (1:50, Serotec, Kidllington, UK); Mac-2 (1:400, Cedarlane, N.C., USA). For fluorescent double-staining of ASL and CD11c the mouse anti ASL (1:50 Santa Cruz Biotechnology, Inc. sc-166787, TX, USA) and rabbit anti CD11c (1:50, ab52632) were used. All antibodies were diluted in PBS containing 2% normal horse serum and 0.2% Triton. Sections were incubated overnight at RT followed by 48 hours at 4° C. Sections were washed three times in PBS and incubated with secondary biotinylated IgG at RT (room temperature) for 1.5 hour, washed three times in PBS and incubated with avidin-biotin Complex (Elite-ABC kit, Vector Lab, CA, USA) at RT for additional 90 minutes followed by DAB (Sigma) reaction. For the fluorescent staining the CY2 conjugated anti mouse and CY3 conjugated streptavidin (1:100 and 1:200 respectively, Jackson ImmunoResearch, West Grove, Pa.) were used.

Tunnel staining was performed by using ApopTag kit detection according to manufacturer's instructions (Millipore, CA, USA). Stained sections were examined and photographed by a fluorescence or bright field microscope (Eclipse Ni-U; Nikon, Tokyo, Japan) equipped with Plan Fluor objectives (10; 20×; 40×) connected to a monochrome camera (DS-Qi1, Nikon).

Nitrite and Nitrate Concentrations in Blood and Colon—

On the day of sacrifice, blood and 1 cm (centimeter) of the distal colon were collected. Blood was separated by centrifuge [1500 RPM (Revolutions per minute) for 5 minutes] to plasma and red blood cells (RBC). Colon tissue was homogenized and proteins in each sample were removed by centrifugation at 10,000 g for 5 minutes following methanol precipitation (colon:methanol=1:2 weight/volume, RBC:methanol=1:1 volume/volume, 4° C.).

Nitrite concentration in the colon and the plasma was measured using a dedicated HPLC system (ENO-20; EiCom, Kyoto, Japan) [22]. This method is based on the separation of nitrite and nitrate by ion chromatography, followed by on-line reduction of nitrate to nitrite, postcolumn derivatization with Griess reagent, and detection at 540 nm (nanometer). Proteins in each sample were removed by centrifugation at 10,000 g for 5 minutes following methanol precipitation (colon:methanol=1:2 weight/volume, plasma:methanol=1:1 volume/volume, 4° C.).

Macrophage Elicitation and Stimulation:

Thioglycollate elicited macrophages were isolated by peritoneal lavage from 5, 8 weeks old mice, in each group. Mice were i.p. injected with 1 ml sterile thioglycollate broth and four days after, peritoneal macrophages were isolated following a previously described method by Zhang et al. (22).

Equal numbers of peritoneal cells were allowed to adhere to cell culture dishes, two plates for each extract, for four hours. Non-adhered cells were removed and adherent cells were washed twice. Cells were cultured in RPMI 1640 arginine free medium containing 20% fetal calf serum (heat inactivated), 1% Penicillin/Streptomycin and 2% L-Glutamine (all from Biological Industries). For each extract, one plate was stimulated with LPS ultrapure (1 μg/ml), for a total of 18 hours or 36 hours while the other plate served as control. Medium was taken for NO analysis and glucose and lactate levels using NOVA. For GC analysis cells were washed with ice cold saline, lysed with 50% methanol in water and quickly scraped followed by three freeze thaw cycles in liquid nitrogen. The insoluble material was pelleted in a cooled centrifuge (4° C.) and the supernatant was collected for consequent GC-MS analysis. Samples were dried under air flow at 42° C. using Techne Dry-Block Heater with sample concentrator (Bibby Scientific Limited, UK) and the dried samples were treated with 40 μl of a methoxyamine hydrochloride solution (20 mg/ml in pyridine) at 37° C. for 90 minutes while shaking followed by incubation with 70 μl N,O-Bis (trimethylsilyl) trifluoroacetamide (Sigma) at 37° C. for additional 30 minutes. the samples were centrifuged and allowed to stand at room temperature for 2 hours before injection.

Gas Chromatography/Mass Spectrometry—

GC/MS analysis was performed using a gas chromatograph (7820AN, Agilent Technologies, USA) interfaced with a mass spectrometer (5975 Agilent Technologies, USA). A HP-5 ms capillary column 30 m×250 μm×0.25 μm (19091S-433, Agilent Technologies, USA) was used. Helium carrier gas was maintained at a constant flow rate of 1.0 mL min-1. The GC column temperature was programmed from 70 to 150° C. via a ramp of 4° C. min-1, 150-215° C. via a ramp of 9° C. min-1, 215-300° C. via a ramp of 25° C. min-1 and maintained at 300° C. for additional 5 minutes. The MS was by electron impact ionization and operated in full scan mode from m/z, 30-500. The inlet and MS transfer line temperatures were maintained at 250° C. and 310° C., respectively. The ion source temperature was 280° C. Sample injection (1-2 μL) was in splitless mode.

Assessment of Plasma Arginine Concentrations—

Blood was collected at the time of sacrifice via cardiac puncture, placed in heparin coated tube and kept on ice. The samples were centrifuged for 5 minutes at 7,000 rpm. The supernatant was removed, aliquoted, snap frozen, and stored at −80° C. until used for amino acid analysis. Analysis was conducted by analytical laboratory services (AMINOLAB LTD).

Complete Blood Count—

Blood was collected at the time of sacrifice via cardiac puncture, placed in EDTA coated tube. Diagnostic Veterinary Pathology Services (Path® Vet).

DNA Extraction from Blood—

QIAamp DNA Blood Mini Kit (Qiagen, Hilden, Germany) (Qiagen, Valencia, Calif.), DNA was isolated from 200 μl of blood according to manufacturer's instruction, in the final step DNA was eluted in 200 μL of buffer AE. The DNA eluate was stored at −20° C. until use in qPCR analysis.

Western Blot—

Western blotting was performed as previously described (Becker systems (Perkin Elmer) and a fluorescence microscope (Olympus, et al., 2003). In some experiments, culture supernatants were used and concentrated by acetone precipitation. Equal amounts of extract (30 or 50 μg) were added to 10 μl electrophoresis sample buffer. After boiling, the proteins were separated by 10% SDS-PAGE, then transferred to nitrocellulose membranes and detected with a specific antibody Anti-Argininosuccinate Lyase (ab97370) and the ECL Western blotting analysis system (Amersham).

FISH Analysis

Tissue Preparation—

Liver tissues were harvested and fixed in 4% paraformaldehyde for 3 hours; incubated overnight with 30% sucrose in 4% paraformaldehyde and then embedded in OCT. 8 μm cryosections were used for hybridization.

Hybridization and Imaging—

Probe libraries were designed and constructed as previously described²³. Single molecule FISH probe libraries consisted of 44-96 probes of length 20 bps, and were coupled to cy5, alexa594 or tmr. Hybridizations were performed overnight in 30° C. DAPI dye for nuclear staining was added during the washes. To detect cell borders alexa fluor 488 conjugated phalloidin (Rhenium A12379) was added to the GLOX buffer wash. Images were taken with a Nikon Ti-E inverted fluorescence microscope equipped with a ×100 oil-immersion objective and a Photometrics Pixis 1024 CCD camera using MetaMorph software (Molecular Devices, Downington, Pa.). The image-plane pixel dimension was 0.13 μm. Quantification of mRNA concentrations were done on stacks of 10 optical sections in the liver, with Z spacing of 0.3 μm. Portal node was identified morphologically on DAPI images based on bile ductule, central vein was identified using smFISH for Glutamine Synthetase performed on serial sections.

Dot Detection—

Dots were automatically detected using previously described algorithms²³′²⁴. Image stacks were filtered with a 3D Laplacian of Gaussian filter with a standard deviation of 1.5 pixels. Next, a range of thresholds was tested, for each threshold the filtered image was converted to a binary image and the number of connected components recorded. The present inventors automatically chose the threshold for which the number of connected component was least sensitive to threshold selection. Each threshold was manually validated and corrected when necessary.

MRI (Magnetic Resonance Imaging)—

Prior to MRI imaging, animals were anesthetized using a mix of Medetomidine/Ketamine. The mouse colon was cleaned using warm water and perfluorinated oil was introduced into the colon via a rectal catheter. MRI experiments were performed on a 9.4 T Bruker BioSpec system using a quadrature volume coil with 35-mm inner diameter. T2 maps were acquired using a multi-slice, spin-echo imaging sequence with the following parameters: repetition delay (TR) of 3000 ms, 16 time-echo increments (linearly spaced from 10 to 160 ms), matrix dimension of 256×128 and two averages, corresponding to an image acquisition time of 12 minutes 48 seconds. Fourteen continuous slices with slice thickness of 1.0 mm were acquired with a field of view of 3.25×2.5 cm². Quantitative T2-maps were generated from multi-echo, T2-weighted images using an in-house Matlab program. The average T2 of the colon was calculated for each slice. Differences in the mean T2 across all of the images slices were compared using a T-test statistic test.

Generation of Bone Marrow (BM) Chimeric Mice—

Villin^{Cre}: As1^{f/f} or As1^{f/f} male mice aged 10-12 weeks were used as recipients. Mice were irradiated with a dose of 950 Gy and reconstituted via IV injection with 5×10⁶ BM cells of either WT or CX₃CR1^{Cre}:I110ra^{fl/fl} mice.

In Vivo Intestinal Permeability Assay—

Assessment of barrier function was performed using an FITC-labeled dextran method. Food and water were withdrawn for 3 hours and mice were orally administrated with permeability tracer (80 mg/100 grams body weight of FITC-labeled dextran, MW 4000; FD4, Sigma-Aldrich). Serum was collected retro-orbitally three hours later and fluorescence intensity was determined (excitation, 492 nm; emission, 525 nm; BioTek). FITC-dextran concentrations were determined using a standard curve generated by serial dilution of FITC-dextran.

Mouse Colon Epithelial and Lamina Propria Isolation—

Colon segment was flushed with cold PBS, longitudinally cut open and rinsed to remove residual luminal contents. Tissue was cut into 3- to 5-mm pieces and incubated in HBSS medium containing EDTA 0.5 M and Hepes 10 mM at 37° C. for 20 minutes, shaken at 180 rpm, to separate epithelial fraction. Supernatant was centrifuged at 4° C. at 500 g for 10 minutes. Recovered tissue was digested in HBSS medium with 2% fetal bovine serum (FBS), 0.05% collagenase II and 0.05% DNase I (Sigma) for 40 minutes. at 37° C., shaken at 180 rpm. LP cells were filtered through 65 μm mesh and centrifuged at 4° C. at 500 g for 10 minutes.

Flow Cytometry—

The present inventors prepared single cell suspensions from mouse colon by enzymatic digestion and analyzed by polychromatic flow cytometry. Samples were stained with conjugated antibodies or matching isotype controls according to manufacturer's instructions. Data were acquired on an LSRII (BD Biosciences) or FACSCalibur instrument (Becton-Dickinson) with CellQuest software, with a MacsQuant instrument (Miltenyi, Bergisch Gladbach, Germany), and analyzed using BD FACSDiva 6 or FlowJo software (version 7.6.5, or version vX.0.7 Tree Star Inc) or MacsQuant. Colon cells were stained for 30 min at 4° C. in flow cytometry buffer (PBS, 10% FCS and 0.02% azide). For antigens that require intracellular staining (As1, iNOS and S-nitrosocystein), cell surface staining was followed by cell fixation and permeabilization with the Cytofix/Cytoperm kit (BD Biosciences) according to the manufacturer's instructions. For staining, the present inventors used a lineage cocktail of antibodies anti-CD45-APC, Anti-CD64-PE, Anti-CD11c-PB, Anti-326-APC cy7, Anti CD31-PE cy7 (all from BioLegend). After staining for cell surface antigens, intracellular NO was detected by incubation of cells for 15 min at 37° C. with 10 μM DAF-FM diacetate followed by extensive washing, according to the manufacturer's instructions (Molecular Probes). Staining without addition of the DAF-FM probe was considered to be the baseline for gating the positive population. As1 was detected using anti-As1-ALEXA FLUOR® 488 (BD Biosciences), iNOS using anti-iNOS followed by anti-rat 488 (Jackson ImmunoResearch), and nitrosylation using anti-S-nitrosocystein (from abcam) followed by anti-mouse 488 (Jackson ImmunoResearch).

Assessment of Colon Epithelial Cells Arginine and Polyamines Concentrations:

The analysis was performed according to a previously described protocol (N. Gray, Plumb, A validated method for the quantification of amino acids in mammalian urine (Waters Corporation, Milford, Mass., 2014) with slight modifications. Briefly, equal number of isolated colon epithelial cells (˜3*10⁶) were incubated in a phosphoric:acetic acid buffer supplemented with 1:50 (v/v; 10 μM) of the following internal standards: hexamethylene diamine and 13C6-arginine (Cambridge Isotope Laboratories). Following 30 minutes at 40° C., cell lysates went through three freeze-thaw cycles using liquid nitrogen.

For the determination of polyamines and amino acids, equal protein 10-μl samples (12 μg) were added to 70 μL of borate buffer (200 mM, pH 8.8 at 25° C.) and mixed. Then, 20 μl of Aqc reagent (10 mM dissolved in 100% ACN) were added and immediately mixed. Aqc reagent was prepared following the procedure described (S. A. Cohen, et al. 1993. Anal. Biochem. 211, 279-287; “Synthesis of a fluorescent derivatizing reagent, 6-aminoquinolyl-N-hydroxysuccinimidyl carbamate, and its application for the analysis of hydrolysate amino acids via high-performance liquid chromatography”). For the determination of proline and arginine, samples were first diluted with the borate buffer 1:10, and then 10 μl aliquots were reacted with Aqc. For derivatization, the samples were heated at 55° C. for 10 minutes, centrifuged at maximum speed for three min and then filtered through a 0.2-μm PTFE filter (Millex-LG, Millipore) to HPLC vials containing inserts.

The LC-MS/MS instrument consisted of Acquity I-class UPLC system and Xevo TQ-S triple quadrupole mass spectrometer (Waters). Chromatographic conditions were used as described in (N. Gray, Plumb 2014, Supra). Mass detection was carried out using electrospray ionization in the positive mode. Argon was used as the collision gas with a flow of 0.1 ml/min. The capillary voltage was set to 3.0 kV, source temperature −150° C., desolvation temperature −650° C., desolvation gas flow—800 L/min, cone voltage 20V. Analytes were detected by monitoring of fragment ion 171 m/z produced from corresponding precursor ions using parameters described in Z. Zwighaft, et al. 2015. (Cell Metab. 22, 874-885. “Circadian clock control by polyamine levels through a mechanism that declines with age”) for polyamines, and in (N. Gray, Plumb 2014, Supra) for amino acids, with exception that collision energy 20 eV was used for regular and labeled 13C6 arginine.

The concentrations were calculated as the response ratio between IS and the analyte using calibration curves of the corresponding compounds. Data processing was performed with TargetLynx software.

ELISA—

For cytokine production measurements, colon tissue single cell suspension was assayed for cytokine levels using a mouse cytokine quantibody array (RayBiotech), according to the manufacturer's instructions. Nitrosilation level was measured using 3-Nitrotyrosine ELISA Kit (abcam), according to the manufacturer's instructions.

Small Molecule Screen Analysis—

MANTRA (F. Iorio, et al. 2010. Proc. Natl. Acad. Sci. 107, 14621-14626; F. Napolitano, et al. 2016., Bioinformatics 32, 235-241) is built upon the Connectivity Map dataset (J. Lamb, et al. 2006. Genes, and Disease, Science 313, 1929-1935) where 1,309 small molecules have been profiled by Affymetrix microarrays on 5 different cell lines at different dosages for a total of 7,000 gene expression profiles (GEPs). MANTRA collapses the 7,000 GEPs into 1,309 Prototype Ranked Lists (PRLs), i.e. one for each small molecule. PRLs represent a consensus rank of differentially expressed genes following treatment with the same drug across multiple cell lines and different dosages. In order to identify the drugs upregulating Ass1 and As1, the present inventors performed a Gene Set Enrichment Analysis (GSEA) on the PRLs for each of 1,309 drugs using the MANTRA online web tool (www(dot)mantra(dot)tigem(dot)it).

16S rRNA Analyses:

Stool sampels of 12 weeks old As1f/f and VillinCre:As1f/f mice on arginine free diet since weaning, and As1f/f mice on standard diet were collected and freez in liquid nitrogen. Samples were processed for DNA isolation using MoBio (PowerSoil kit) according to the manufacturer's instructions. The purified DNA from feces was used for PCR amplification and sequencing of the bacterial 16S rRNA gene. Amplicons of −380 base pairs spanning the variable region 3-4 (V3-4) of the 16S rRNA gene were generated by using designated primers. The PCR products were subsequently pooled in an equimolar ratio, purified (PCR clean kit, Promega), and used for Illumina MiSeq sequencing. Reads were processed using the QIIME (quantitative insights into microbial ecology) analysis pipeline as described(48) version 1.8. Paired-end joined sequences were grouped into operational taxonomic units (OTUs) using the UCLUST algorithm and the GreenGenes database(49). Sequences with distance-based similarity of 97% or greater over median sequence length of 353 bp were assigned to the same OTU. Analysis was performed at each taxonomical level (phylum to genus and specie level if possible) separately. For each taxon, statistical tests were performed between the different groups. P values are FDR-corrected for multiple hypothesis testing.

Statistics:

All statistical analyses were performed using one-way ANOVA, two-way ANOVA, repeated-measures ANOVA or Student's t-test. Contrasts comparing groups with one specific treatment or control were tested with a Dunnett's test. Log-transformed data were used where differences in variance were significant and variances were correlated with means. Sample sizes were chosen in advance on the basis of common practice of the described experiment and are mentioned for each experiment. No statistical methods were used to predetermine sample size. Each experiment was conducted with biological and technical replicates and with three replications of the entire experiment unless specified otherwise. Statistical tests were done using Statsoft's STATISTICA, version 10. All error bars are standard errors. P<0.05 was considered significant in all analyses (* P<0.05, ** P<0.005, *** P<0.0005). A survival analysis, accounting for genotype and batch, was conducted using a Cox proportional hazards model (using the ‘survival’ package in R).

### Example 1

### The Role of Asl in Colitis is Cell Dependent

### Experimental Results

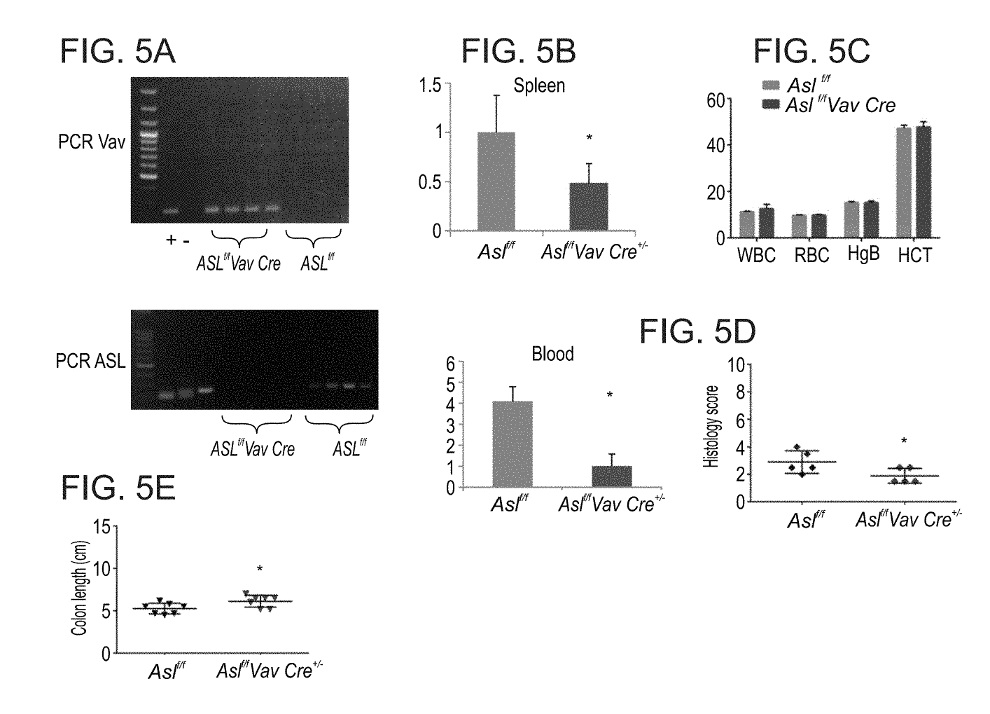

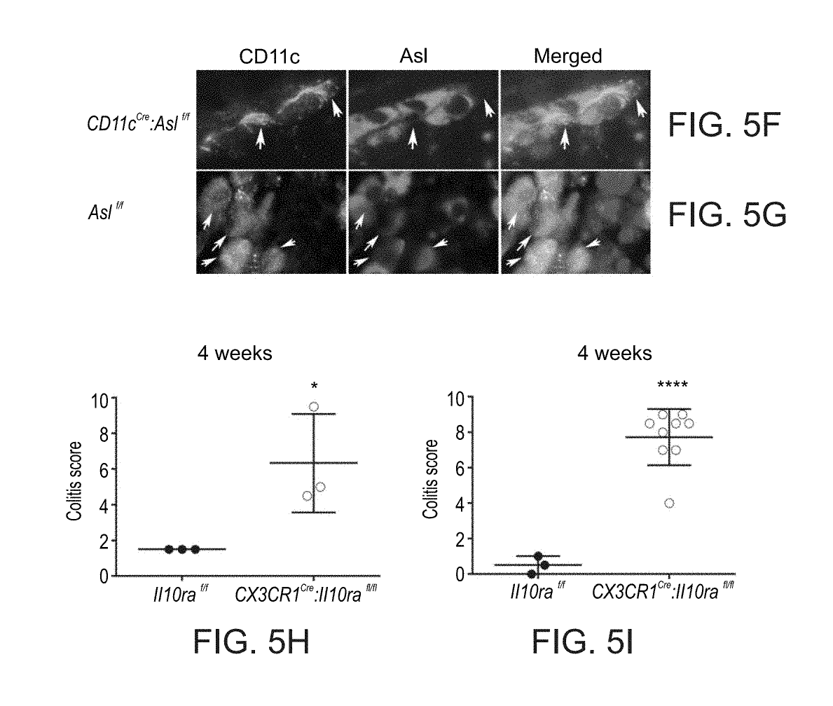

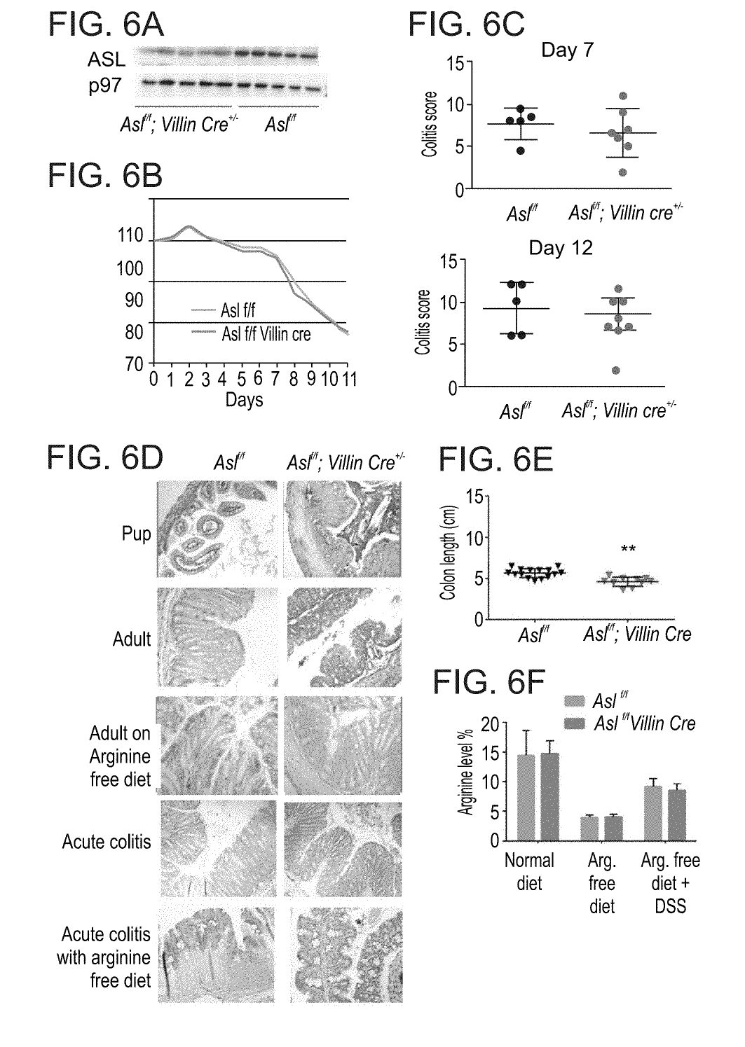

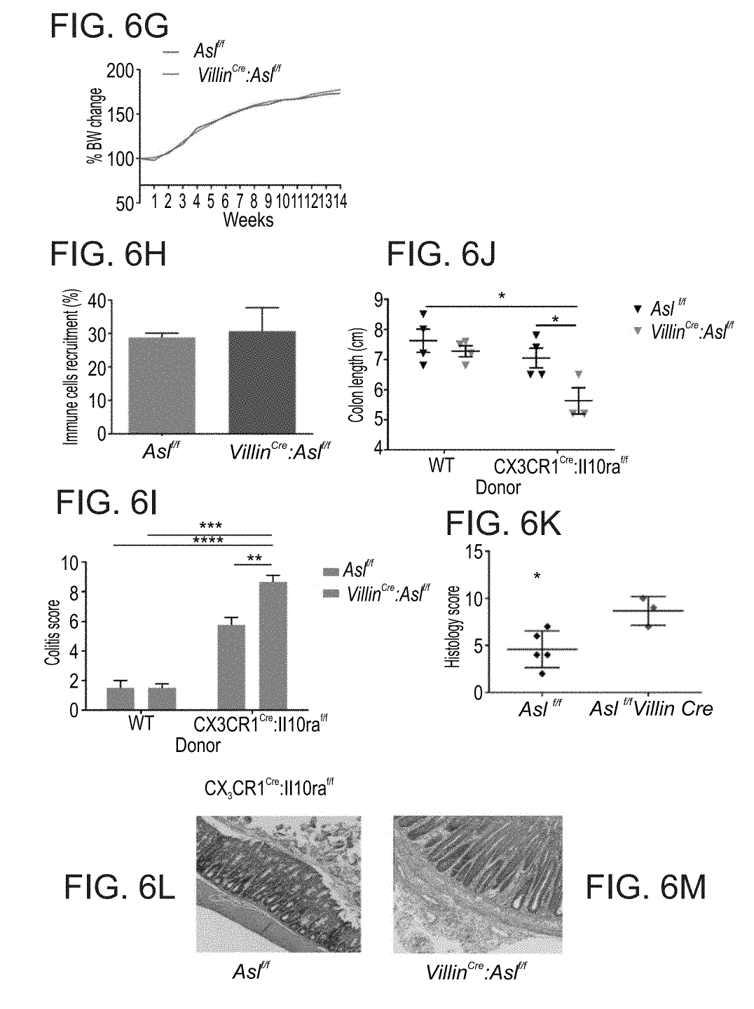

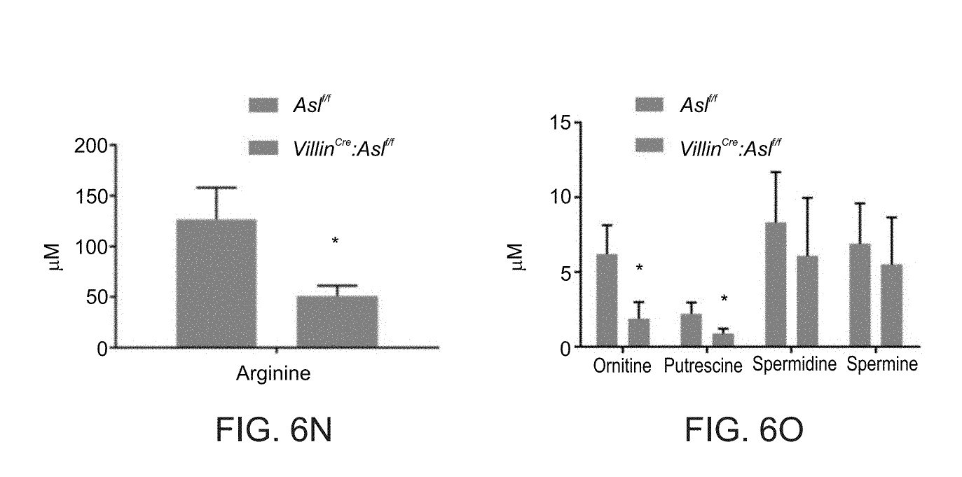

To generate cell-specific conditional knockout (CKO) mice of ASL the present inventors crossed As1^{f/f} animals (Erez, A. et al. 2011, Nat. Med. 17, 1619-1626) to transgenic mice expressing Cre recombinase under the enterocyte-specific Villin-(Madison, B. B. et al. 2002. J. Biol. Chem. 277, 33275-33283), the hematopoietic Vav1-promoter (Ogilvy, S. et al., 1998, Blood 91, 419-430) and under the macrophage/dendritic cell (DC)-specific CD11c-promoter (Caton, M. L. et al. 2007. J. Exp. Med. 204, 1653-1664; Vander Lugt, B. et al. 2014. Nat. Immunol. 15, 161-167) (FIGS. 1A-D and FIGS. 5A-E). For the epithelial deletion of As1, the present inventors used the As1^{f/f};Villin cre^{tg/+} mice previously generated and characterized as described elswhere⁷ (FIG. 1A). At baseline, all CKO mice were indistinguishable from their wild-type (WT) littermates and demonstrated no observable phenotype; in particular, there were no differences in intestinal histology and blood counts (Data not shown and FIGS. 2C and 6D). The present inventors induced colitis in all As1 conditional knockouts using the established dextran sulfate sodium (DSS) model⁸ (chemical induction) or by genetic methods using IL-10 receptor-deficient myeloid immune cells. Specifically, the present inventors had previously shown that a macrophage-specific deletion of the receptor for the anti-inflammatory cytokine IL-10 results in spontaneous and chronic gut inflammation (Zigmond et al., 2014), mirroring the situation in pediatric patients with IL-10 receptor subunit alpha (IL-10Rα), mutations (20). The colitis disease progression was comprehensively evaluated both clinically as well as by endoscopic evaluation, histology and MRI imaging. In addition, the present inventors measured colon length and scored colitis severity histologically postmortem.

In an extension of this model, lethally irradiated WT animals that received a bone marrow (BM) graft from CX₃CR1^{Cre}:I110ra^{f/f} mice developed spontaneous colitis and hence this procedure was used by us to generate a genetic model for IBD (FIGS. 5H-I). The severity of intestinal inflammation after induction of colitis was assessed comprehensively by using endoscopy, histology magnetic resonance imaging (MRI), as well as by evaluating weight and survival.

The present inventors recently found that As1 deletion in enterocytes increased the propensity of newborn mice to develop enterocolitis⁷. Surprisingly, when the present inventors used the same As1^{f/f} Villin cre^{tg/+} mice but in adulthood as a model for IBD, these finding could not be recapitulated (FIGS. 6A-C). The discrepancy was confirmed to be due to the documented abolishment of ASL expression in the enterocytes after weaning when there is enough arginine in the diet to supply the body needs⁸ (FIG. 6D). Meaning, the lack of difference in colitis severity in adult mice between the control and the transgenic mice was because the control mice did not express As1 at sufficiently different levels as compared to the As1^{f/f} Villin cre^{tg/+}.

To test whether it is possible to elevate As1 levels in control mice, the present inventors fed the mice with arginine free diet and found no difference in growth curves and life span between As1^{f/f} Villin cre^{tg/+} and controls, but the growth rate in both groups was about 20% lower as compared to mice fed with standard diet (Data not shown). Encouragingly, the present inventors found that arginine free diet is able to upregulate As1 expression in enterocytes, suggesting that As1 can be regulated in the adult intestine in arginine deficient states (FIG. 6D). Indeed, colitis induction in As1^{f/f} Villin cre^{tg/+} mice receiving arginine free diet, is more severe in comparison to control mice, as measured by colitis scores, colon length, weight loss and MRI imaging (FIGS. 1B-F and FIG. 6E). Furthermore, the augmented colitis severity decreases the survival of As1^{f/f} Villin cre^{tg/+} mice (FIG. 1G). Dietary arginine restriction resulted in expression of ASL in the intestines of control mice and not the Villin^{Cre}:As1^{f/f} animals however, no differences in the growth curves or immune cell recruitment to the intestine were observed between the CKO and controls (FIGS. 6G-O). Collectively, these results suggest that ASL induction in the adult mouse gut is a physiological mechanism to restore arginine homeostasis during arginine deficient states.

In contrast and counter intuitively, As1^{f/f} Vav cre^{tg/+} mice in which As1 is deleted in their immune cells, had reduced colitis severity in comparison to control mice by all measured parameters; these mice had decreased colitis score as evaluated by colonoscopy, histologically and by MRI imaging and in concordance, had significantly longer colons. Importantly, these mice were less sick clinically as exemplified by losing less weight and having normal hemoglobin levels while the control group developed anemia and extramedullary hematopoiesis likely due to blood loss in the stool (FIGS. 2A-B and FIGS. 5C-E).

Together, these results suggest that depletion of As1 in a cell specific manner results in differences in colitis severity; supporting a protective role for NO production by the enterocytes and a potentially an injurious role for NO secreted by the immune cells, increasing colonic injury. Importantly, these results show that enterocytes are able to re-express a functional As1 in arginine deficient states.

### Example 2

### The Role of Endogenous No During Colitis is Different in Enterocytes Vs. Immune Cells

To understand the increased colitis pathogenicity in mice with As1 knockout in the enterocytes when given arginine free diet, the present inventors first measured arginine levels at homeostasis on both standard or arginine free diet, and while under colitis induction.

In human and murine enterocytes, expression of ASL is maximal in the second week of life, when the intestine is the principal site of arginine synthesis (21). In contrast, enterocyte expression of ASL in adult mice is rather limited and thus expectedly, was not significantly different as compared to the Villin^{Cre}:As1^{f/f} mice (FIGS. 5F-G).

Interestingly, under all these conditions the present inventors found that As1 deficiency in the enterocytes did not cause a significant difference in plasma arginine levels between the As1^{f/f};Villin cre^{tg/+} and control mice (FIG. 6F).

With arginine free diet, induction of colitis with the standard dose of DSS was lethal in the Villin^{Cre}:As1^{f/f} mice, thus necessitating that a lower dose of DSS (0.8%) be used for all experiments. Even with this reduced DSS challenge, the increased severity of colitis in the Villin^{Cre}:As1^{f/f} was evident by the findings of greater weight loss, higher colonic inflammation as assessed by endoscopy, decreased colon length by histology, decreased intestinal relaxation time on MRI (a surrogate marker of inflammation (Martin, D. R. et al., 2005. Top. Magn. Reson. Imaging 16, 77-98), higher histological score, and finally decreased survival (FIGS. 1B-G), as compared to control mice. Indeed, following DSS-induced colitis, ASL levels in the control enterocytes were not elevated significantly; the severity of DSS-induced colonic inflammation was similar in the controls and Villin^{Cre}:As1^{f/f} mice (FIGS. 6B, 6D, 6F). These results are consistent with previous work by the present inventors that showed increased incidence of necrotizing enterocolitis in Villin^{Cre}:As1^{f/f} mice only in the neonatal period when there is a significant expression of As1 in WT enterocytes.

In elucidating the source of arginine synthesis, the present inventors found a compensatory upregulation of As1 RNA levels in the liver of As1^{f/f};Villin cre^{tg/+} mice (FIG. 1G). These results suggest that at baseline and during colitis, arginine is synthesized by As1 in the intestine or the liver. Interestingly, in spite of the lack of differences in systemic arginine levels, the present inventors found decreased NO levels in the intestine of the As1^{f/f};Villin cre^{tg/+} mice as compared to control mice (FIG. 1H). This result is in concordance with the structural requirement for As1 protein in addition to its catalytic function in generating arginine, for the formation of NO synthesis complex⁶. Hence, while ASL is essential systemically for the generation of arginine, it is required locally and explicitly in enterocytes during colitis, for arginine production as substrate for NO. When the enterocytes cannot upregulate As1, the liver takes over and upregulates As1 which elevates arginine levels, but cannot compensate for the inability to synthesize NO in the enterocytes where it is required. Indeed, analysis of publically available RNA expression levels of biopsies taken from human with IBD, show upregulation of As1 in IBD patients intestine as compared to its levels in the intestine of controls (FIG. 1I).

To determine whether the decreased colitis severity following As1 deletion in immune cells by the Vav transgenic Cre is also attributed to NO, the present inventors measured NO biomarker levels in the red blood cells (RBC) and in the colon before and after colitis induction. While the present inventors found a significant increase in the total NO level in RBC and colon after colitis induction in the control group, there was no significant change in NO levels in the As1^{f/f} Vav cre^{tg/+} mice RBC or colon, suggesting an autocrine and paracrine role for NO produced by the immune cells respectively (FIG. 2D). Since the major immune cells that produce NO are the macrophages (MQ)¹², the present inventors isolated MQ from As1^{f/f} Vav cre^{tg/+} mice and measured their response to lipopolysaccharide (LPS) to recapitulate inflammatory state. Similar to the finding at the whole organism level, the present inventors found that As1^{f/f} Vav cre^{tg/+} MQs produce less NO in response to LPS (FIG. 4E). Recently, it has been shown that in addition to the paracrine role of NO secreted by the MQ on other cells, it has an cell autonomous function in activating the MQ to M1 state metabolically, by promoting a “Warburg effect” with inhibition of oxidative phosphorylation and induction of glycolysis¹³. Indeed, the present inventors found that decreasing NO production by the MQ in the As1^{f/f} Vav cre^{tg/+} mice, results in decreased MQ activation as exemplified by no change in lactate and glucose levels in response to LPS. In contrast, MQ isolated from control mice and activated by LPS, show increase in NO production which induces the switch towards glycolytic metabolism as demonstrated by decreased glucose and increase in lactate levels (FIG. 2F). In parallel, citrate which provides metabolites necessary for NO production¹⁴, is elevated in the MQ isolated from the As1^{f/f} Vav cre^{tg/+}, most likely due to their inability to utilize it for NO synthesizes (FIG. 2G). Furthermore, histological assessment showed decreased activated MQ recruitment in vivo to the intestine during colitis (FIG. 2H). Thus, knockout of As1 in immune cells in general and in the MQ specifically, results in decreased NO production leading to decreased MQ activation which manifests as decreased colitis severity. These results together suggest that NO secreted by the MQ during colitis enhances the intestinal tissue damage.

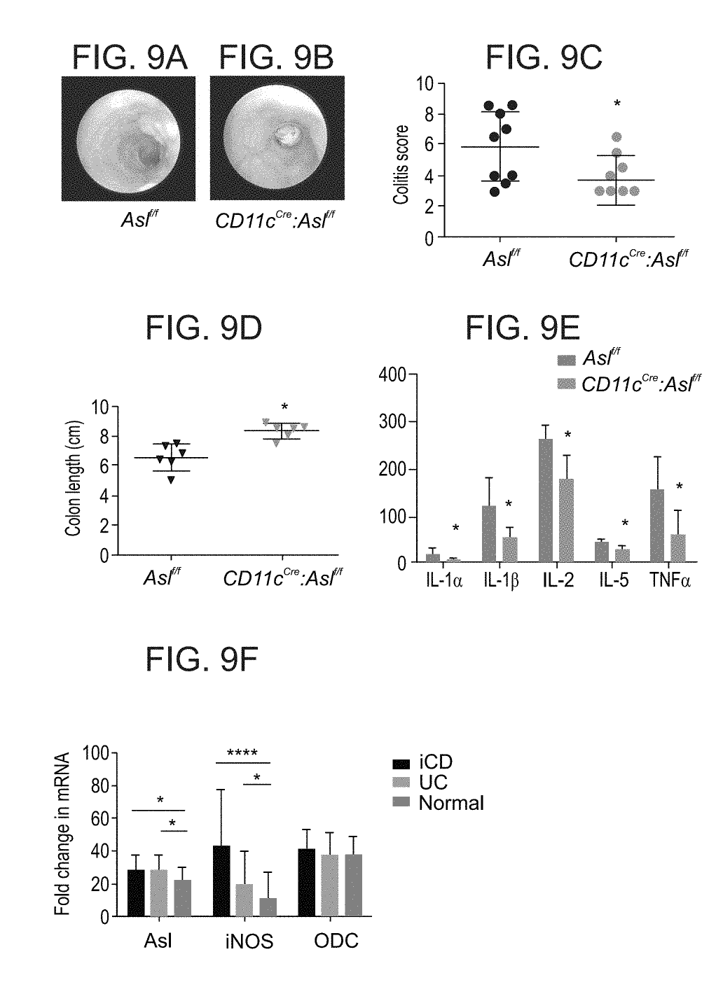

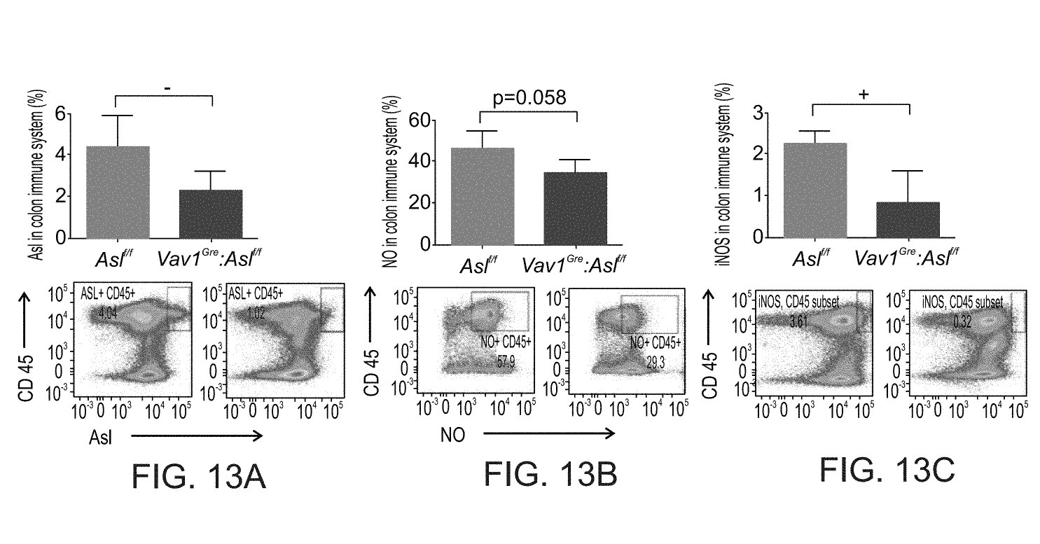

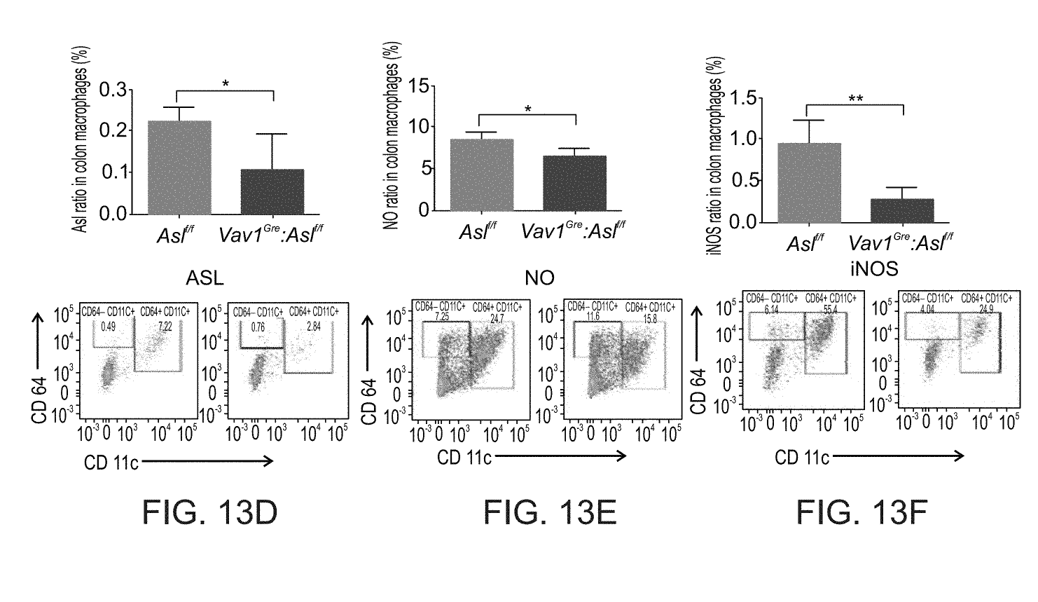

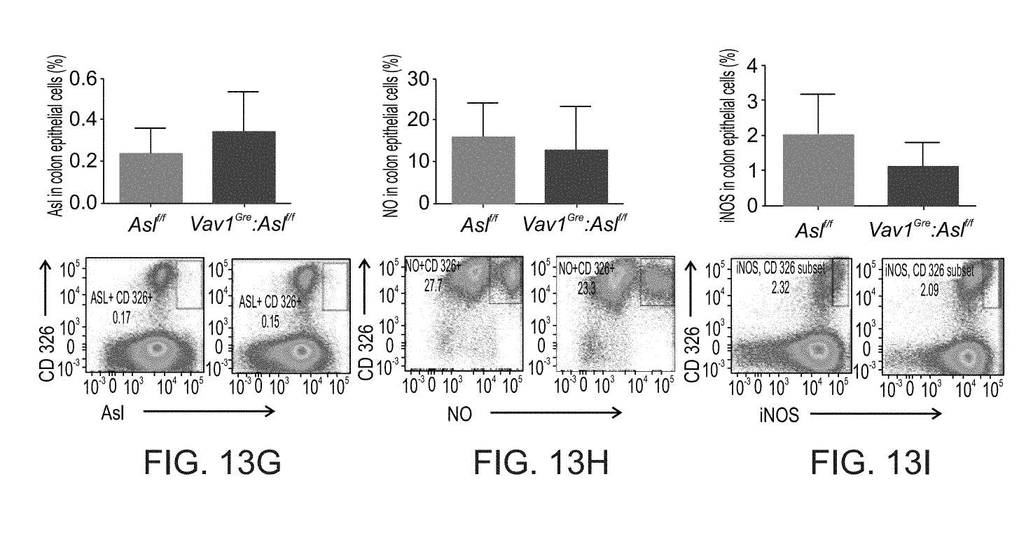

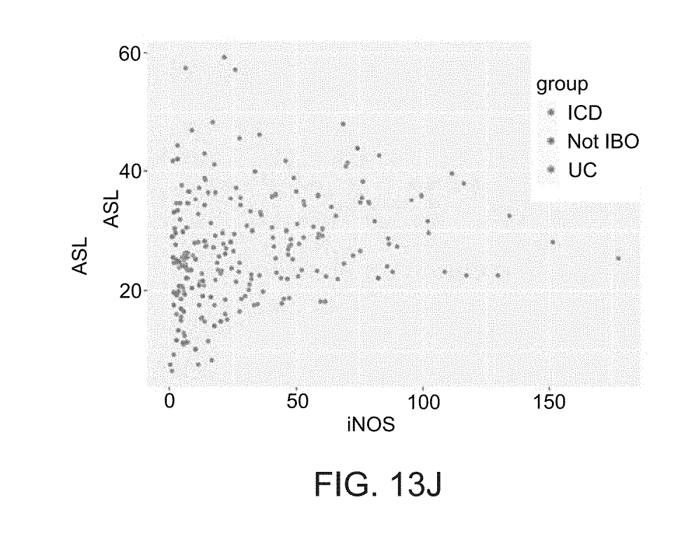

To further investigate the intestinal macrophages, the present inventors analyzed the levels of iNOS and NO in intestinal immune cells (CD45+) and enterocytes (CD326+). As expected, the present inventors found ASL levels to be significantly lower in intestinal CD45+ cells of immune ASL deficient mice, correlating with decreased iNOS expression and decreased NO production; in contrast, there were no significant differences in these parameters in the enterocytes (FIGS. 13A-J). To corroborate these results, the present inventors induced colitis in CD11c^{Cre}:As1^{f/f} mice and found a decrease in weight loss, colitis scores and pro-inflammatory cytokines, and an increase in colon length, all supporting ameliorated colitis (FIGS. 9A-C). Hence, NO produced by the intestinal immune cells exacerbates the immune response, which contributes to the injurious effects during colitis.

Collectively, without being bound by any theory, these data suggest that loss of ASL in the enterocytes has a significant impact on increasing the severity of inflammation, while loss of ASL in the immune cells alleviates colitis severity, likely due to changes in NO levels. Interestingly, analysis of the GEO data base revealed that ASL expression is increased in chronic IBD patients with Crohn or Ulcerative colitis as compared to controls and moreover, in contrast to ODC expression, ASL expression correlates with iNOS expression (FIG. 9D and FIG. 13J). This analysis may further support the present inventors' findings for the importance of ASL re-expression in enterocytes during colitis for NO synthesis, and the relevance of these models to human IBD.

Colitis Induction by Genetic Modification—

To prove that enterocyte loss of ASL increases colitis severity independent of the colitis model used, the present inventors induced colitis in As1^{f/f} and Villin^{Cre}:As1^{f/f} by bone marrow (BM) transplantation from CX₃CR1^{Cre}:I110ra^{f/f} donor mice. Similarly to DSS induced colitis, on arginine-free diet, colitis driven by the pro-inflammatory IL10R deficient macrophages in Villin^{Cre}:As1^{f/f} was exacerbated compared to control mice, as evident by higher colitis and histology score, as well as decreased colon length (FIGS. 6I-J).

Collectively, without being bound by any theory, these results of two distinct colitis models suggest that colitis severity is at least in part, driven by arginine availability specifically in the enterocytes during arginine-deficient states.

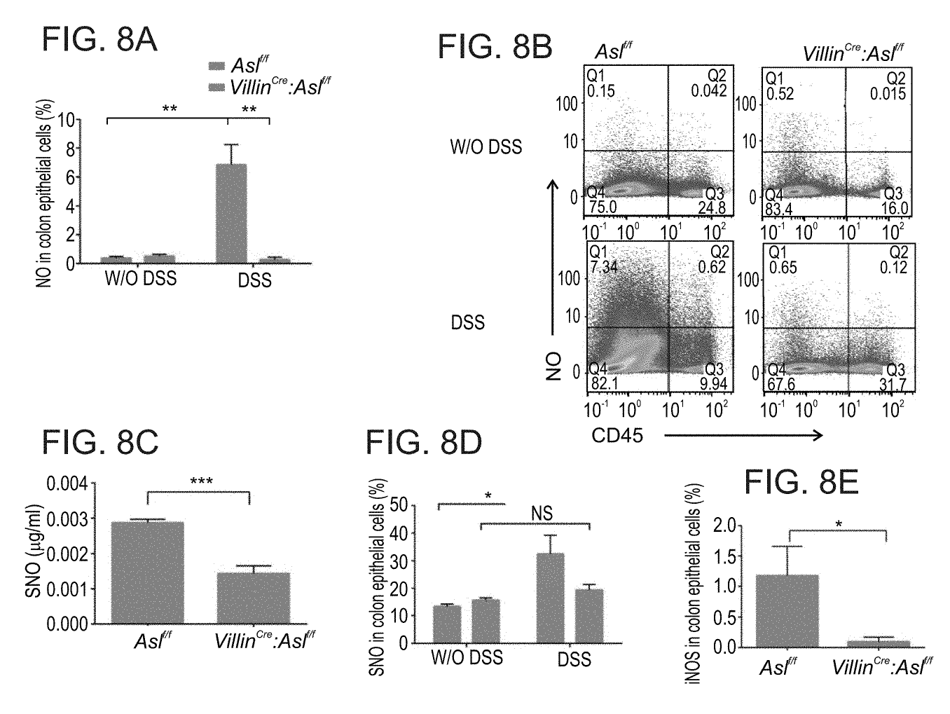

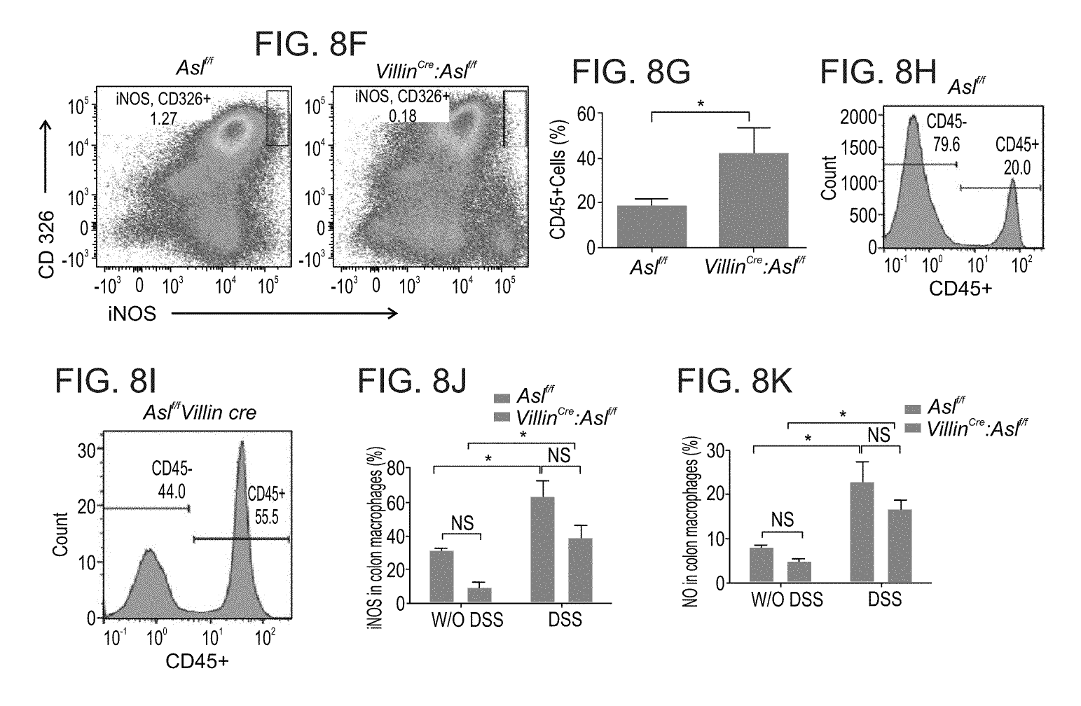

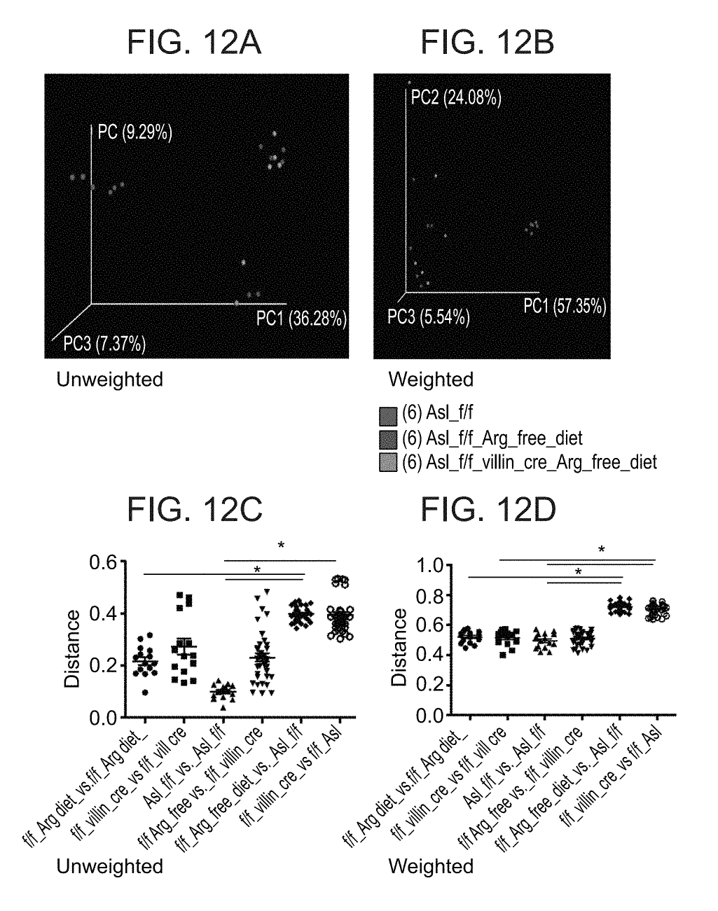

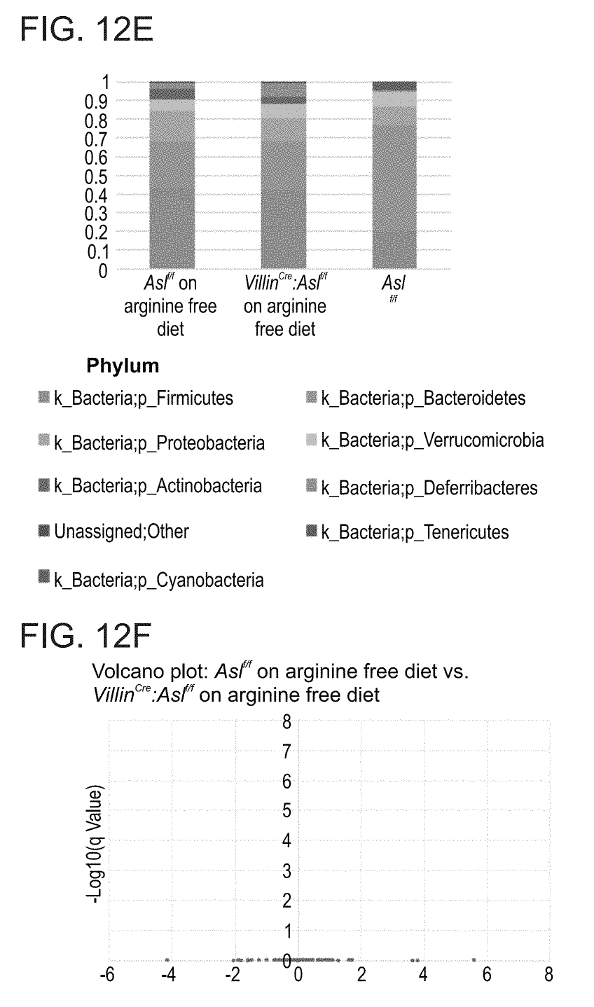

The Increased Colitis Severity Associated with ASL Loss in the Enterocytes is Mediated by NO—

Whereas plasma arginine in Villin^{Cre}:As1^{f/f} and control mice were similar, the combination of arginine restricted diet and colitis induction resulted in significantly reduced arginine levels in the enterocytes of the Villin^{Cre}:As1^{f/f} mice (FIGS. 6A-O). These results suggested that cell-specific deficiency of arginine and potentially one of its downstream metabolites could be mediating inflammation severity. As the present inventors had previously shown a critical role for ASL in NO metabolism (A. Erez, et al., 2011, Nat. Med. 17, 1619-1626), the present inventors wanted to assess whether the enterocyte-specific arginine deficiency led to decreased NO synthesis. The present inventors measured nitrite levels as a biomarker of NO in the intestines of Villin^{Cre}:As1^{f/f} animals and controls mice 10 days after induction of colitis. This time point was chosen as at this time with DSS-induced colitis, the intestine is in post-inflammatory regenerative phase (C. G. Whittem, et al., 2010, JoVE J. Vis. Exp. e1652-e1652). Thus, nitrite measurements from intestinal homogenates are expected to be more representative of NO production from the enterocytes, rather than immune cells. In the enterocytes of the Villin^{Cre}:As1^{f/f} mice the present inventors found significantly decreased NO levels, as well as reduced nitrosylation and decreased iNOS levels, likely due to the specific decrease in arginine levels (FIGS. 8A-F, FIG. 6D). Simultaneously, during colitis there was increased recruitment of immune cells to the intestine of the Villin^{Cre}:As1^{f/f} animals (FIGS. 8G-I). Of note, recruited macrophages in the Villin^{Cre}:As1^{f/f} mice were phenotypically similar to cells infiltrating the intestine of controls, as they were able to upregulate iNOS expression and to generate NO in response to DSS at similar levels (FIGS. 8J and 8K). Since arginine is also the substrate for polyamine synthesis via ornithine, and since polyamines were shown to play a role in colitis, the present inventors measured the intestinal levels of these metabolites as well. While levels of the arginine downstream metabolites ornithine and putrescine were lower in Villin^{Cre}:As1^{f/f} mice, there was no significant difference in the levels of spermidine and spermine or in the permidine/spermine ratio which was found to be critical for normal growth and development(28). Importantly, microbiome sequence analysis confirmed that without colitis and on arginine free diet, there were no significant differences in the microbiome composition between the Villin^{Cre}:As1^{f/f} mice and controls (FIGS. 12A-F). Collectively, these results highlight the enterocyte requirement of arginine during colitis in the specific compartment, where NO is synthesized. Furthermore, these data allude to the possibility that an enterocyte-restricted NO deficiency might contribute to immune cell recruitment to the gut and hence exacerbate inflammation.

### Example 3

### Inducing Endogenous No Production by Enterocytes Metabolically Alleviates Colitis

The present inventors next wanted to see if these findings could have translational relevance for therapy in colitis, by testing whether it is possible to metabolically upregulate NO levels specifically in enterocytes.

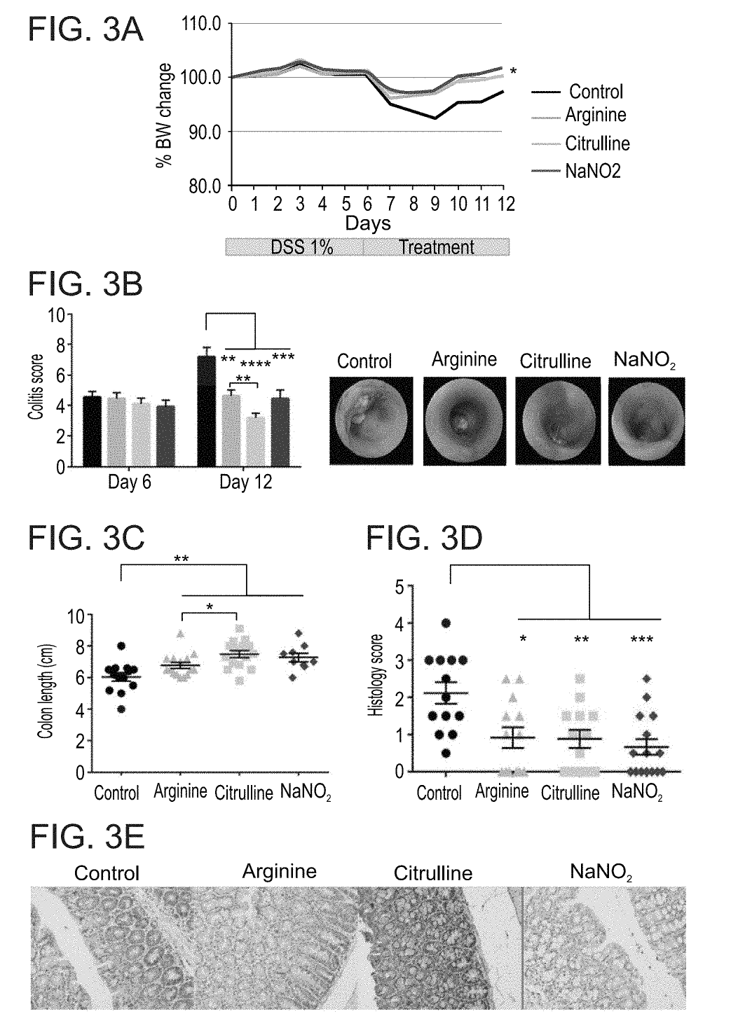

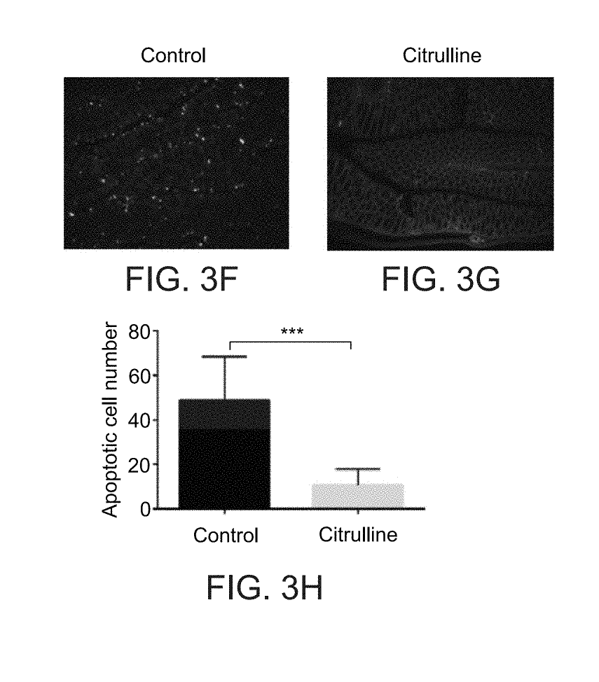

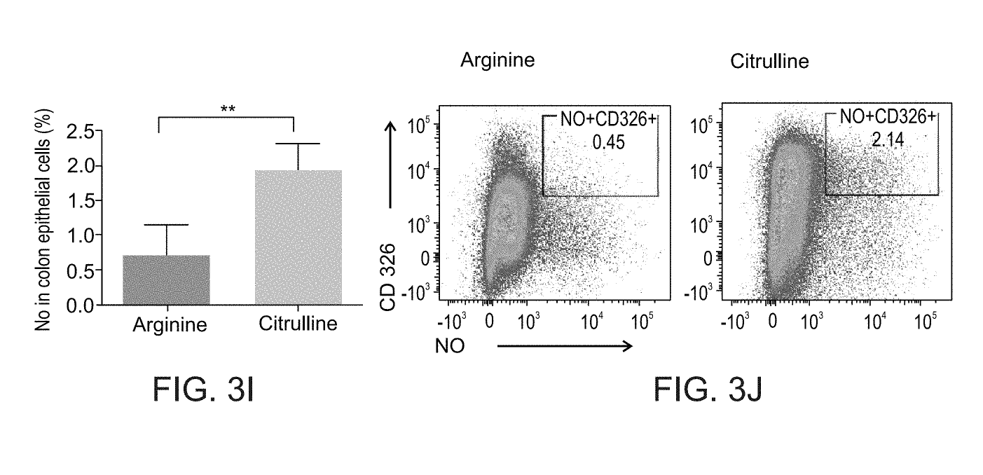

To evaluate potential treatment modalities related to the arginine/NO flux, the present inventors evaluated the therapeutic effect of supplementing arginine, citrulline and the NO donor sodium nitrate, on colitis severity in wild type mice. Indeed, all three drugs led to a significant clinical improvement in colitis severity as evaluated by change in weight, histological score and colon length, with citrulline being the most effective (FIG. 3A-D). Furthermore, while citrulline and arginine upregulated As1 levels in the intestine as compared to control mice with colitis, NO donors did not, suggesting a synergistic beneficial effect between supplementing the substrates for NO generation and the upregulation of As1 (FIG. 3E). Importantly, citrulline was most beneficial in alleviating colitis severity in most parameters when compared to arginine or NO donors (FIG. 3B-C), likely because arginine and NO donors have multiple fates. In addition, there are gaps in the knowledge regarding the therapeutic kinetics of NO related drugs as well as in the ability to accurately target NO to a specific cell; preventing from maximizing its therapeutic potential¹⁶. Citrulline however, drives the synthesis of arginine for NO generation¹⁵ and indeed, analysis of apoptosis level in the intestine of mice treated with citrulline, showed decreased apoptosis, explaining the decreased colitis severity (FIGS. 3F-3H).

Furthermore, intestines of mice treated with citrulline, showed increased generation of NO in the enterocytes and decreased apoptosis correlating with decreased colitis severity (FIGS. 3F-J). Thus, further studies to explore enterocyte-specific induction of ASL and NO were conducted using citrulline.

Finally, to prove beyond reasonable doubt that it is the enterocytes' expression of As1 that is responsible for the protective and alleviating the phenotype that the present inventors find in colitis, the present inventors used Mantra software to search for FDA approved small molecules that upregulate As1¹⁷ (Table 3, hereinbelow). The present inventors found that in multiple cell lines (cancer cell lines), the flavanol molecule fisetin (3, 7, 3′, 4′-tetrahydroxyflavone) significantly upregulates the RNA expression levels of both enzymes required for arginine generation, As1 and Ass1, and was hence chosen by the present inventors to be tested for its ability to promote the NO cycle metabolically during colitis in vivo. Fisetin is commonly found in many fruits and vegetables and has been shown to have an anti-inflammatory property (B. D. Sahu, et al. 2016. J. Nutr. Biochem. 28, 171-182). The present inventors were hence interested to understand its specific effect on enterocytes. Encouragingly, supplementing the mice with fesitin upregulated As1 and Ass1 expression in the enterocytes and improved colitis severity in all parameters (FIG. 4A-E).

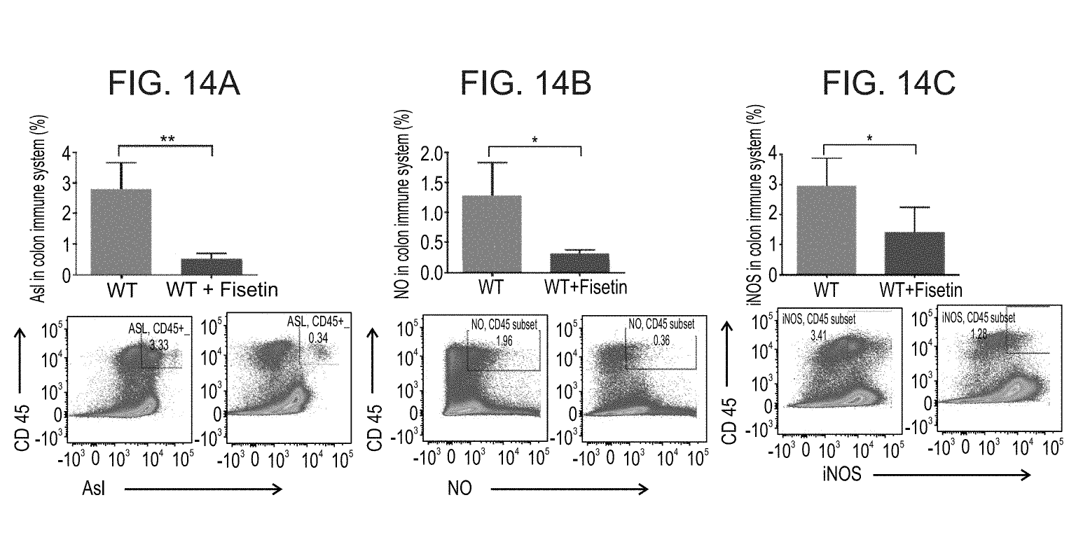

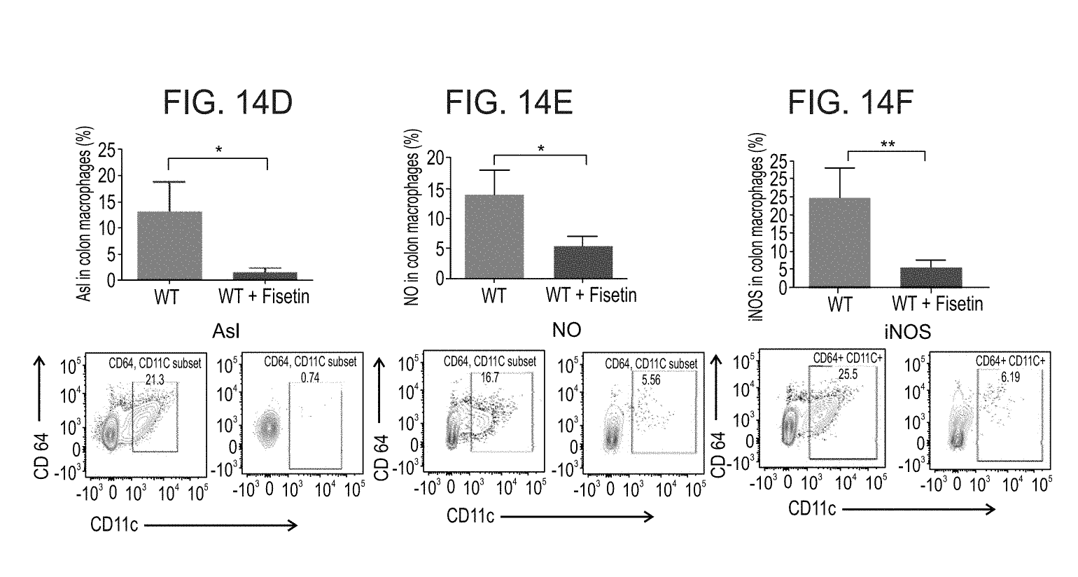

Moreover, the increase in ASL levels was specific to the enterocytes, and correlated with increased iNOS expression and NO production levels in the enterocytes and not in the intestinal immune cells (FIGS. 4H-L and FIGS. 14A-F). Importantly, fisetin therapy improved colitis severity in WT mice in all parameters as compared NO donors; yet it did not have an effect on the enterocyte-specific ASL CKO, suggesting that it is the specific upregulation of ASL in the intestinal epithelium that is beneficiary against colitis (FIG. 4M-N).

Since citrulline is the upstream metabolic substrate for ASS1, the present inventors expected that combining it with fisetin, will help to achieve the most prominent affect. Indeed, supplementing mice with fisetin which increases Ass1 and As1 expression together with the substrate citrulline, was the most prominent modality (a synergistic effect) to generate NO in the enterocytes, and was hence the most effective treatment for colitis in both the chemically and genetically induced colitis models (FIGS. 4B-G and 10A-D).

TABLE 3
Table 3: FDA approved small molecules that affect Asl and Ass1
expression levels. The table summarizes all FDA approved small
molecules that upregulate or downregulate Asl and Ass1 expression
levels in response to FDA approved small molecular screen.

| Gene | Up | Down |
|---|---|---|
| ASL | Alsterpaullone | Prestwick-1083 |
| | Copper sulfate | Baclofen |
| | Dexamethasone | Butirosin |
| | Fisetin | Chlorpropamide |
| | | clofazimine |
| | | Doxepin |
| | | Isosorbide |
| | | Pinacidil |
| | | Pyridoxine |
| | | Thiocolchicoside |
| | | Trazodone |
| ASS1 | azacitidine | Cefotaxime |
| | Fistein | celecoxib |
| | Staurosporine | |

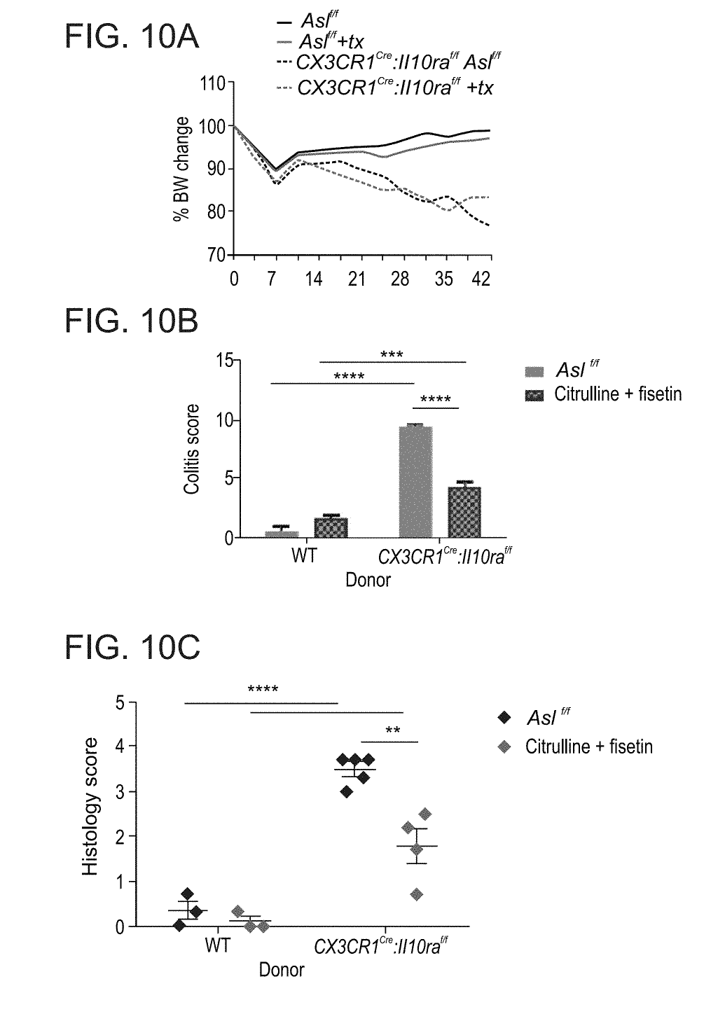

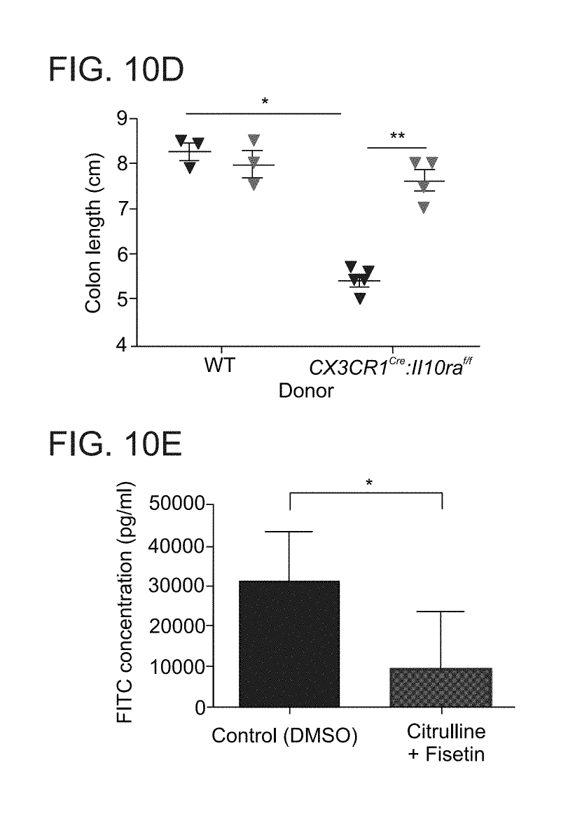

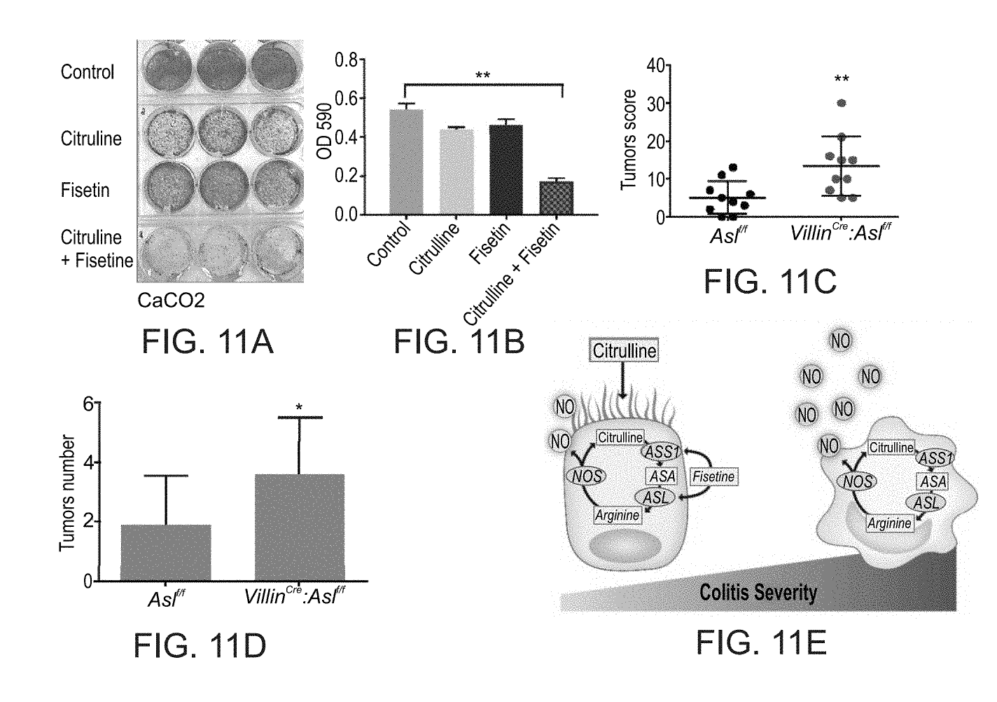

The present inventors further demonstrated the improved epithelial integrity following increased NO production by fisetin and citrulline in the genetic model of colitis by measuring significantly decreased plasma levels of FITC conjugated dextran (FIG. 10E). To confirm the human relevance of these findings, the present inventors measured conductivity in Caco2 human epithelial cells following exposure to DSS and different drug regimens. The present inventors next plated the Caco2 cells as monolayer, to enable their growth as colorectal carcinoma, and encouragingly saw growth restriction following the combined treatment (FIGS. 11A-B). As colitis severity has been shown to predispose to colon cancer development, the present inventors used the DSS-Azoxymethane (AOM) model, for colitis associated cancer. While there was no significant difference in cancer severity between As1^{f/f} and Vav1^{Cre}: As1^{f/f}, (Data not shown), Villin^{Cre}: As1^{f/f} had a higher tumor burden, reflecting more and larger tumors (FIGS. 11C-D).

Collectively, without being bound by any theory, these results show that NO levels can be metabolically modulated specifically in the enterocytes by regulation of ASL function, which increases endogenous NO production at the precise amount and dosage as needed to strengthen the epithelial barrier and hence mitigate colitis and decrease the associated chronic complications.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Tones, M. I. & Rios, A. Current view of the immunopathogenesis in inflammatory bowel disease and its implications for therapy. World J. Gastroenterol. WJG 14, 1972 (2008).
2. Baumgart, D. C. & Carding, S. R Inflammatory bowel disease: cause and immunobiology. The Lancet 369, 1627-1640 (2007).
3. Blaise, G. A., Gauvin, D., Gangal, M. & Authier, S. Nitric oxide, cell signaling and cell death. Toxicology 208, 177-192 (2005).
4. Suschek, C. V., Schnorr, 0. & Kolb-Bachofen, V. The role of iNOS in chronic inflammatory processes in vivo: is it damage-promoting, protective, or active at all? Curr. Mol. Med. 4, 763-775 (2004).
5. Kolios, G., Valatas, V. & Ward, S. G. Nitric oxide in inflammatory bowel disease: a universal messenger in an unsolved puzzle. Immunology 113, 427-437 (2004).
6. Erez, A. et al. Requirement of argininosuccinate lyase for systemic nitric oxide production. Nat. Med. 17, 1619-1626 (2011).
7. Premkumar, M. H. et al. Argininosuccinate lyase in enterocytes protects from development of necrotizing enterocolitis. Am. J. Physiol.-Gastrointest. Liver Physiol. 307, G347-G354 (2014).
8. de Jonge, W. J., Dingemanse, M. A., de Boer, P. A. J., Lamers, W. H. & Moorman, A. F. M. Arginine-Metabolizing Enzymes in the Developing Rat Small Intestine. Pediatr. Res. 43, 442-451 (1998).
9. Azad, N. et al. S-Nitrosylation of Bc1-2 Inhibits Its Ubiquitin-Proteasomal Degradation A NOVEL ANTIAPOPTOTIC MECHANISM THAT SUPPRESSES APOPTOSIS. J. Biol. Chem. 281, 34124-34134 (2006).
10. Jayasooriya, R. G. P. T. et al. Antagonistic effects of acetylshikonin on LPS-induced NO and PGE2 production in BV2 microglial cells via inhibition of ROS/PI3K/Akt-mediated NF-κB signaling and activation of Nrf2-dependent HO-1. Vitro Cell. Dev. Biol.-Anim. 51, 975-986 (2015).
11. Zhang, H. et al. Glucagon□like peptide□1 protects cardiomyocytes from advanced oxidation protein product□induced apoptosis via the PI3K/Akt/Bad signaling pathway. Mol. Med. Rep. (2015). doi: 10.3892/mmr.2015.4724
12. Lorsbach, R. B., Murphy, W. J., Lowenstein, C. J., Snyder, S. H. & Russell, S. W. Expression of the nitric oxide synthase gene in mouse macrophages activated for tumor cell killing. Molecular basis for the synergy between interferon-gamma and lipopolysaccharide. J. Biol. Chem. 268, 1908-1913 (1993).
13. Kelly, B. & O'Neill, L. A. Metabolic reprogramming in macrophages and dendritic cells in innate immunity. Cell Res. 25, 771-784 (2015).
14. A critical role for citrate metabolism in LPS signalling I Biochemical Journal. Available at: www.biochemj.org/content/438/3/e5.abstract. (Accessed: 12 Mar. 2016)
15. Wijnands, K. A. P. et al. Citrulline a More Suitable Substrate than Arginine to Restore NO Production and the Microcirculation during Endotoxemia. PLoS ONE 7, e37439 (2012).
16. Scatena, R., Bottoni, P., Martorana, G. E. & Giardina, B. Nitric oxide donor drugs: an update on pathophysiology and therapeutic potential. Expert Opin. Investig. Drugs 14, 835-846 (2005).
17. Iorio, F. et al. Discovery of drug mode of action and drug repositioning from transcriptional responses. Proc. Natl. Acad. Sci. 107, 14621-14626 (2010).
18. Luiking, Y. C., Engelen, M. P. K. J. & Deutz, N. E. P. REGULATION OF NITRIC OXIDE PRODUCTION IN HEALTH AND DISEASE. Curr. Opin. Clin. Nutr. Metab. Care 13, 97-104 (2010).
19. Kelm, M. Nitric oxide metabolism and breakdown. Biochim. Biophys. Acta BBA—Bioenerg. 1411, 273-289 (1999).
20. Wirtz, S., Neufert, C., Weigmann, B. & Neurath, M. F. Chemically induced mouse models of intestinal inflammation. Nat. Protoc. 2, 541-546 (2007).
21. Becker, C., Fantini, M. C. & Neurath, M. F. High resolution colonoscopy in live mice. Nat. Protoc. 1, 2900-2904 (2006).
22. Gonçalves, R. & Mosser, D. M. The isolation and characterization of murine macrophages. Curr. Protoc. Immunol. 14-1 (2008).
23. Itzkovitz, S. et al. Single-molecule transcript counting of stem-cell markers in the mouse intestine. Nat. Cell Biol. 14, 106-114 (2012).
24. Lyubimova, A. et al. Single-molecule mRNA detection and counting in mammalian tissue. Nat. Protoc. 8, 1743-1758 (2013).

1. (canceled)

2. A method of treating inflammatory bowel disease (IBD) and/or colon cancer comprising administering to a subject in need thereof a therapeutically effective amount of citrulline and a therapeutically effective amount of a flavonoid, thereby treating the subject.

3. A pharmaceutical composition comprising a therapeutic effective amount of citrulline and a therapeutically effective amount of a flavonoid for the treatment of an inflammatory bowel disease (IBD) and/or colon cancer.

4. (canceled)

5. The method of claim 2, wherein said citrulline is co-administered with a therapeutically effective amount of a flavonoid.

6. The method of claim 2, wherein said therapeutically effective amount of said citrulline and/or of said flavonoid upregulates the level of nitric oxide in enterocyte cells of the subject.

7. The method of claim 2, wherein said therapeutically effective amount of said citrulline and/or of said flavonoid upregulates the level of Argininosuccinate Lyase (ASL) in enterocyte cells of the subject.

8. The method of claim 7, wherein said flavonoid is naturally-occurring flavonoid.

9. The method of claim 8, wherein said naturally-occurring flavonoid is fisetin.

10. The method of claim 2, wherein said IBD comprises ulcerative colitis.

11. The method of claim 2, wherein said IBD comprises Crohn's disease.

12. The method of claim 2, wherein said disease is colon cancer.

13. The method of claim 2, further comprising administering to the subject an agent which downregulates Argininosuccinate Lyase (ASL) in immune cells of the subject.

14. The method claim 13, wherein said immune cells comprise macrophages.

15. The pharmaceutical composition of claim 3, wherein said flavonoid is fisetin.

16. The pharmaceutical composition of claim 3, wherein said IBD comprises ulcerative colitis.

17. The pharmaceutical composition of claim 3, wherein said IBD comprises Crohn's disease.

18. The pharmaceutical composition of claim 3, wherein said disease is colon cancer.


SEQUENCE LISTING (7 sequences)

SEQ ID NO: 1 - protein, 464 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSHAVALTRDSERLLEVRKRINVLPLGSGAIAGNPLGVDRELLRAELNFGAITLNSMDATSERDFVAEFLFWASLCMTHLSRMAEDLILYCTKEFSFVQLSDAYSTGSSLMPQKKNPDSLELIRSKAGRVFGRCAGLLMTLKGLPSTYNKDLQEDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

SEQ ID NO: 2 - DNA, 2061 bp, Homo sapiens
ccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 3 - DNA, 1937 bp, Homo sapiens
gacgccatcccggccagaaaagccctggccagtggcgggcgcgacactatccgtgcggccaggcggagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 4 - protein, 444 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSHAVALTRDSERLLEVRKRINVLPLGSGAIAGNPLGVDRELLRAELNFGAITLNSMDATSERDFVAEFLFWASLCMTHLSRMAEDLILYCTKEFSFVQLSDAYSTGSSLMPQKKNPDSLELIRSKAGRVFGREDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

SEQ ID NO: 5 - DNA, 2001 bp, Homo sapiens
ccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcgggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 6 - protein, 438 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSGAIAGNPLGVDRELLRAELNFGAITLNSMDATSERDFVAEFLFWASLCMTHLSRMAEDLILYCTKEFSFVQLSDAYSTGSSLMPQKKNPDSLELIRSKAGRVFGRCAGLLMTLKGLPSTYNKDLQEDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

SEQ ID NO: 7 - DNA, 1983 bp, Homo sapiens
ccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa